(19) United States
(12) Patent Application Publication
(10) Pub. No.: US 2020/0230067 A1
(43) Pub. Date: Jul. 23, 2020

(54) SUSPENSIONS OF ENCAPSULATED PHARMACEUTICALS AND METHODS OF MAKING AND USING THE SAME

(71) Applicant: Panacea Biomatx, Inc., Research Triangle Park, NC (US)

(72) Inventors: Lloyd Staton Noel, Research Triangle Park, NC (US); Edison Thurman Hudson, Research Triangle Park, NC (US); Scott Alexander Baldwin, Research Triangle Park, NC (US); Charles Allen Queen, Research Triangle Park, NC (US); John Daniel Schneible, Research Triangle Park, NC (US)

(21) Appl. No.: 16/835,715

(22) Filed: Mar. 31, 2020

Related U.S. Application Data
(63) Continuation of application No. PCT/US18/54116, filed on Oct. 3, 2018.
(60) Provisional application No. 62/567,779, filed on Oct. 4, 2017.

(51) Int. Cl. A61K 9/50; A61K 31/616; A61K 31/40; A23P 10/30; A23L 33/15

(52) U.S. Cl. CPC A61K 9/5031; A61K 31/616; A23V 2002/00; A23P 10/30; A23L 33/15; A61K 31/40

(57) ABSTRACT

The presently disclosed subject matter is directed a system and method of creating personalized doses of active pharmaceutical ingredients (APIs) dispersed in a palatable oral formulation. The APIs are encapsulated into microparticles that are dispersed within a thixotropic suspension vehicle to create a customized oral formulation. The formulation can be customized for a particular subject based on medical condition, time release of the API, release profile of the API, and taste preference of the subject.

### CROSS-REFERENCE TO RELATED APPLICATIONS

This application is a continuation of U.S. Patent Application No. PCT/US18/54116, filed on Oct. 3, 2018, which claims priority to U.S. Provisional Patent Application No. 62/567,779, filed Oct. 4, 2017, the entire contents of which are all hereby incorporated herein by reference.

### TECHNICAL FIELD

The presently disclosed subject matter relates to oral liquid suspensions of encapsulated pharmaceuticals for the personalized treatment of one or more medical conditions.

### BACKGROUND

Two of the biggest problems facing the health care system are prescription non-adherence and “one size fits all” pharmaceutical formulations. The prevailing system for pharmaceutical treatment is to prescribe numerous pills and/or liquids of fixed doses to patients, oftentimes without feed-forward information about the patient's medical history and personal biology. The patient is then relied upon to follow confusing daily and weekly medical regimens. This problem is known as “the pill burden”, and is attributed to one of every twenty deaths in the United States. Problematically, the pill burden problem is exacerbated in populations that struggle with pill consumption (e.g., pediatrics and geriatrics), which are often the populations that need treatment the most. Thus, it would be beneficial to provide improved pharmaceutical consumption systems to overcome the cited challenges.

### SUMMARY

In some embodiments, the presently disclosed subject matter is directed to a suspension for oral consumption. The suspension comprises a plurality of microparticles, wherein each microparticle comprises a core and an external coating surrounding the core. The core comprises about 20-99 weight percent of at least one active pharmaceutical ingredient (API), based on the total weight of the core; about 0.1-10 weight percent disintegrant, based on the total weight of the core; and about 0.1-10 weight percent monosaccharide, polysaccharide, or both, based on the total weight of the core. The suspension further comprises a thixotropic suspension media, wherein the suspension media is homogeneously distributed with the microparticles, and wherein the core, coating, and suspension media prevent the API from releasing into the suspension until ingestion by a user. In some embodiments, the microparticles are homogeneously suspended within the suspension media for at least 5, 10, 15, 20, 24, 48, or 36 hours after suspension preparation.

In some embodiments, the monosaccharide or polysaccharide is selected from sucrose, fructose, maltose, cellobiose, lactose, trehalose, lactulose, glucose, ribose, galactose, dextrose, talose, arabinose, fucose, mannose, xylose, erythrose, starch, glycogen, cellulose, or combinations thereof.

In some embodiments, the suspension media is a hydrocolloid or oleogel.

In some embodiments, the suspension is a semi-solid.

In some embodiments, the suspension comprises one or more different types of microparticles, each comprising a different API. In some embodiments, the suspension comprises a different API and/or a different excipient.

In some embodiments, the microparticles are evenly distributed within the suspension media.

In some embodiments, the suspension media comprises dextrose, sucrose, fructose, maltose, cellobiose, lactose, trehalose, lactulose, glucose, ribose, galactose, dextrose, talose, arabinose, fucose, mannose, xylose, erythrose, starch, glycogen, cellulose, or combinations thereof in an amount of about 50 mM to about 500 mM. Thus, the suspension media can comprise a monosaccharide and/or polysaccharide in a concentration of at least about (or no more than about) 50 nM, 100 mM, 150 mM, 200 mM, 250 mM, 300 mM, 350 mM, 400 mM, 450 mm, or 500 mM.

In some embodiments, the suspension allows modified release, immediate release, delayed release, or extended release of at least one API.

In some embodiments, the microparticles are evenly distributed within the suspension media.

In some embodiments, the API remains primarily partitioned in the microparticles after elevated temperature pasteurization, food additive pasteurization, or both.

In some embodiments, the suspension allows for release of less than about 5% of the API in the suspension while stored. Thus, the suspension can provide for the release of less than about 5%, 4%, 3%, 2% or 1% of the API in suspension while stored (e.g., prior to ingestion by the user).

In some embodiments, the API is selected from one or more pharmaceuticals, vitamins, or food supplements.

In some embodiments, the coating is selected from hydroxypropyl methylcellulose, sodium carboxymethylcellulose, cellulose acetate, hydroxypropylcellulose, povidone, cellulose acetate phthalate, methyl hydroxyethylcellulose, ethylcellulose, gelatin, pharmaceutical glaze, plasticizer, hydroxypropyl cellulose, hydroxypropyl methyl cellulose phthalate, cellulose acetate phthalate, polyvinyl acetate phthalate, methacrylic acid copolymer, methylcellulose, polyethylene glycol, polyvinyl acetate phthalate, shellac, sucrose, titanium dioxide, polyvinyl polymers, acrylate polymers, ethyl cellulose, cellulose acetate, wax, zein, or combinations thereof.

In some embodiments, the coating comprises one or more layers.

In some embodiments, the coating comprises about 1-50 weight percent of the microparticle, based on the total weight of the microparticle.

In some embodiments, the suspension further comprises at least one additive selected one or more surfactants, colorants, dispersants, preservatives, taste improvers, flavorings, sweeteners, antioxidants, or combinations thereof.

In some embodiments, the suspension comprises about 30-99 weight percent suspension media and about 1-70 weight percent microparticles, based on the total weight of the suspension.

In some embodiments, the microparticles have an average particle size of between about 100-1000 microns.

In some embodiments, the presently disclosed subject matter is directed to a method of preparing a suspension comprising a uniform dispersion of microencapsulated active pharmaceutical ingredients (APIs). Particularly, the method comprises receiving health-related information for a subject, determining an API to treat a medical condition of the subject, and selecting microparticles of a desired API. Each microparticle includes a core and a coating, wherein the core comprises about 20-99 weight percent of at least one active pharmaceutical ingredient, based on the total weight of the core; about 0.1-10 weight percent disintegrant, based on the total weight of the core; and about 0.1-10 weight percent monosaccharide, polysaccharide, or both, based on the total weight of the core. The method further comprises determining a thixotropic hydrocolloid suspension media (e.g., a semisolid thixotropic hydrocolloid filler medium), and dispersing a predetermined amount of the microparticles within the suspension media to form a dosage. The suspension media is solubilized to embed the microparticles and is then reformed as a homogeneously distributed semi-solid suspension. The core, coating, and suspension media prevent the API from releasing into the suspension until ingestion by a user.

In some embodiments, the filler medium is a hydrocolloid or oleogel.

In some embodiments, the suspension allows modified release of at least one API. In some embodiments, the API is selected from one or more pharmaceuticals, vitamins, or food supplements.

In some embodiments, the coating is selected from hydroxypropyl methylcellulose, sodium carboxymethylcellulose, cellulose acetate, hydroxypropylcellulose, povidone, cellulose acetate phthalate, methyl hydroxyethylcellulose, ethylcellulose, gelatin, pharmaceutical glaze, plasticizer, hydroxypropyl cellulose, hydroxypropyl methyl cellulose phthalate, cellulose acetate phthalate, polyvinyl acetate phthalate, methacrylic acid copolymer, methylcellulose, polyethylene glycol, polyvinyl acetate phthalate, shellac, sucrose, titanium dioxide, polyvinyl polymers, acrylate polymers, ethyl cellulose, cellulose acetate, wax, zein, or combinations thereof.

In some embodiments, the coating comprises one or more layers.

In some embodiments, the microparticles have an average particle size of between about 100-1000 microns.

In some embodiments, the presently disclosed subject matter is directed to a method of treating a medical condition. The method comprises administering a therapeutically effective amount of one or more active pharmaceutical ingredients (APIs) to a subject in need thereof. The API is configured in a suspension for oral consumption. The suspension comprises a uniform dispersion of microparticles. Each microparticle includes a core and a coating, wherein the core comprises about 20-99 weight percent of at least one active pharmaceutical ingredient, based on the total weight of the core; about 0.1-10 weight percent disintegrant, based on the total weight of the core; and about 0.1-10 weight percent monosaccharide, polysaccharide, or both, based on the total weight of the core. The method comprises receiving health-related information for a subject; determining an API to treat a medical condition of the subject; selecting microparticles of a desired API; determining a semisolid thixotropic hydrocolloid suspension media; dispersing a predetermined amount of the microparticles within the suspension media to form a dosage; wherein the suspension media is solubilized to embed the microparticles and is then reformed (e.g., as a semi-so. The core, coating, and suspension media prevent the API from releasing into the suspension until ingestion by a user.

In some embodiments, the API is taste masked (e.g., the taste, flavor, and/or texture of the API is masked or hidden by the suspension media).

In some embodiments, the medical condition is a chronic condition, such as (but not limited to) cancer, type II diabetes, rheumatoid arthritis, or cardiovascular disease.

In some embodiments, the microparticle is capable of remaining in the small intestine for at least about 5 hours and releases API during at least part of the residence time.

In some embodiments, the suspension comprises a volume of about 0.25-10 ounces. In some embodiments, the suspension comprises a volume of about 1-4 ounces.

In some embodiments, the therapeutically effective amount of API is configured based on a dosing requirement of the subject.

### BRIEF DESCRIPTION OF THE DRAWINGS

The previous summary and the following detailed descriptions are to be read in view of the drawings, which illustrate some (but not all) embodiments of the presently disclosed subject matter.

FIGS. 1a, 1b, and 1c are three embodiments of API microparticles in accordance with some embodiments of the presently disclosed subject matter.

FIGS. 2a-2c are particle size distribution graphs of API microparticles constructed in accordance with some embodiments of the presently disclosed subject matter.

FIGS. 3a and 3b are SEM images at 100× and 250×, respectively, of API microparticles comprising an aspirin core produced in accordance with some embodiments of the presently disclosed subject matter.

FIG. 3c is a cross-sectional SEM image (413×) of an API microparticle with a coated aspirin core in accordance with some embodiments of the presently disclosed subject matter.

FIGS. 3d and 3e are SEM images at 100× and 250× of an API microparticle with a coated Atorvastatin core in accordance with some embodiments of the presently disclosed subject matter.

FIG. 3f is a SEM image of an API with a coated Atorvastatin core at 800× magnification in accordance with some embodiments of the presently disclosed subject matter.

FIG. 4 is a line graph illustrating the dissolution of a coated aspirin API microparticle over time.

FIG. 5a is a plot of aspirin core API microparticles released over time at pH 6 and 7.3.

FIG. 5b is a plot of coated aspirin core API microparticles released over time at pH 6 and 7.3.

FIG. 6 is a plot of a coated aspirin core API microparticle release profile over time at pH 2, 4, and with water.

FIG. 7 is a plot of the release profile of coated aspirin core API microparticles in no salt, low salt, and high salt storage buffer.

FIG. 8a is plot of the release profile of coated aspirin core API microparticles in no sugar, low sugar, and high sugar storage buffer at pH 2.

FIG. 8b is plot of the release profile of coated aspirin core API microparticles in no sugar, low sugar, and high sugar storage buffer at pH 4.

FIG. 9 is a plot of the release profile of coated aspirin core API microparticles at no sugar or high sugar intermediate storage solution.

FIG. 10a is a release profile of coated aspirin core API microparticles in citric acid buffer at pH 2 in the presence and absence of sucrose and xanthan gum.

FIG. 10b is a release profile of coated aspirin core API microparticles in citric acid buffer at pH 4 in the presence and absence of sucrose and xanthan gum.

FIGS. 11a-11e are SEM images (50×) of API microparticles with disintegrant added to the core at 0 minutes, 30 seconds, 2 minutes, 5 minutes, and 10 minutes after the addition of water.

FIGS. 12a-12d are SEM images (50×) of API microparticles in the absence of disintegrant at 0 minutes, 30 seconds, 2 minutes, 5 minutes, and 10 minutes after the addition of water.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a coating” can include a plurality of such coatings, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

The presently disclosed subject matter is directed to a system and method of creating personalized doses of active pharmaceutical ingredients (APIs) dispersed in a palatable oral formulation. The APIs are encapsulated into microparticles that are dispersed within a thixotropic suspension vehicle to create a customized oral formulation. The term “microparticle” refers to a particle having a particle size in the micron-sized range, or from 0.1 microns to about 1000 microns. In some embodiments, when the microparticle is substantially spherical in shape, the particle size refers to the diameter of the microparticle (e.g., in the micron-sized range). In embodiments where the microparticle does not have a spherical shape, the particle size can refer to the equivalent diameter of the particle relative to a spherical particle or can refer to a dimension (length, breadth, height or thickness) of the non-spherical particle. The microparticle can have any desired shape, such as spherical, abstract, etc.

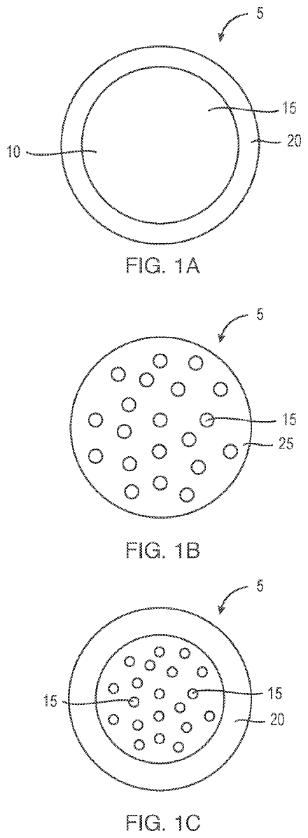

In some embodiments, the disclosed microparticles are of the reservoir type (e.g., marked by one or more film coatings surrounding an inner core of API material, also called a “core shell microparticle”), as shown in FIG. 1a. Particularly, microparticle 5 comprises core 10 comprising API 15 that is encapsulated by outer coating 20. The term “core” refers to the central or innermost portion of the microparticle. Alternatively, the disclosed microparticles can be of the matrix type (e.g., marked by an inhomogeneous single layer wherein API 15 is dispersed throughout excipient 25 and there is no film-coating), as shown in FIG. 1b. In some embodiments, the disclosed microparticles are a combination of a matrix particle and a reservoir particle, such as a matrix core with one or more film coatings 20, as shown in FIG. 1c.

Suitable APIs that can be included within the disclosed microparticles can comprise pharmaceuticals, vitamins, food supplements, and combinations thereof that can be orally administered. For example, suitable pharmaceuticals can include any of the wide variety of orally-administered chemical compounds that can be used for prevention, diagnosis, treatment, and/or cure of a medical condition. In some embodiments, the pharmaceutical can be used to treat a chronic condition, such as (but not limited to) cardiovascular disease, type 2 diabetes, rheumatoid arthritis, and/or some forms of cancer. Vitamins suitable for packaging within the disclosed microparticles can include (but are not limited to) thiamine, riboflavin, niacin, nicotinic acid, pantothenic acid, pyridoxine, biotin, folic acid, vitamin B₆, vitamin B₁₂, lipoic acid, vitamin C, vitamin A, vitamin D, vitamin E, vitamin K, and derivatives thereof. Food supplements suitable for inclusion within the disclosed microparticles can include any of the wide variety of ingestible compositions that affect the response of the body to a food and/or enhance the quality of a food, such as (but not limited to) minerals, antioxidants, botanicals, amino acids, and combinations thereof. For example, in some embodiments the food supplement can be selected from the group comprising iron, calcium, selenium, iodine, magnesium, BHT, BHA, flavonoids, beta carotene, polyphenol, glutathione, Echinacea, flaxseed, gingko, turmeric, L-arginine, L-glutathione, L-lysine, and combinations thereof. It should be understood that optional ingredients can further be included within the disclosed microparticles, such as (but not limited to) flavorings and/or colorings.

The disclosed microparticles can include about 5-95 weight percent API, based on the total weight of the microparticle. The microparticle can therefore comprise at least about (or no more than about) 5, 10, 15, 20, 25, 30, 35, 40, 45, 50, 55, 60, 65, 70, 75, 80, 85, 90, or 95 weight percent API, based on the total weight of the microparticle.

In some embodiments, the APIs are capable of fully dissolving in aqueous solutions, lipid solutions, or both. In some embodiments, the microparticle coating is pH-dependent, thereby controlling release of the APIs positioned within the microparticle core. Particularly, in some embodiments, the microparticle coating allows release of the core APIs at a pH of greater than about 4 (e.g., at least about 4.5, 5, 5.5, or 6). For example, in some embodiments, the pH of the suspension media comprising the coated API microparticles can be maintained at about 4 or less. As a result of the low pH, the APIs remain within the microparticle cores and are not released into the suspension media. In some embodiments, about 5% or less (e.g., about 5, 4, 3, 2, or 1% or less) of the APIs are released from the microparticle core during storage in the suspension media. After ingestion by a patient (e.g., eating or drinking), the suspension passes through the stomach at a pH of about 1-3. Due to the low pH, the APIs are maintained within the microparticle core. Once the suspension passes to/through the upper and lower intestines (e.g., duodenum) with a pH of about 5-8, the microparticle coating is dissolved, allowing for the release of the APIs from the microparticle core.

In some embodiments, the APIs can be micronized. The term “micronized” as used herein refers to a particle size in the micrometer range, e.g., a particle size from 0.1 to 100 μm. Particles can be micronized using any method known or used in the art, such as milling, grinding, precipitation, rapid expansion of supercritical solutions, spray drying, fractionation, filtration, sol-gel processes, spray reaction synthesis, flame synthesis, liquid foam synthesis, prilling, atomization, emulsion, and the like.

The selected API can be in the free form or can be in the form of a salt, ester, hydrate, solvate, polymorph, isomer, or any other pharmaceutically acceptable forms, as would be known to those of ordinary skill in the art. In some embodiments, suitable APIs can be between about 10-500 μm in size. For example, in some embodiments, pre-processed micronized APIs can be about 10-200 μm in size and pre-processed APIs (e.g., using a wax-prilling technique) can be about 100-500 μm in size. However, the presently disclosed subject matter is not limited and can include embodiments where the APIs are larger or smaller than the ranges given above.

In some embodiments, the microparticle can include one or more APIs and at least one disintegrant. The term “disintegrant” as used herein refers to a material added to a dosage form to help it break apart (disintegrate) and release the API. Suitable distintegrants can include (but are not limited to) microcrystalline celluloses and cross-linked celluloses such as (sodium croscamnellose), starches, modified starches (such as sodium carboxymethyl starch, croscarmellose sodium starch, sodium starch glycolate), natural and synthetic gums (such as locust bean, karaya, guar, tragacanth, and agar), cellulose derivatives (such as methylcellulose and sodium carboxymethylcellulose), alginates (such as alginic acid and sodium alginate), clays (such as bentonites), and effervescent mixtures. The amount of disintegrant in the microparticle can range from about 0.01% to about 15% by weight (e.g., 0.1-15%, 2-12%, or 3-10%). Thus, the microparticle can include at least about (or no more than about) 0.01, 0.1, 0.5, 1, 2, 3, 4, 5, 6, 7, 8, 9, 10, 11, 12, 13, 14, or 15 weight % disintegrant, based on the total weight of the microparticle. It should also be appreciated that in some embodiments, the disclosed microparticles lack disintegrant.

In some embodiments, the microparticle can include one or more polysaccharides and/or monosaccharides. Monosaccharides are carbohydrates that cannot be hydrolyzed to simpler compounds. Polysaccharides are carbohydrates that can be hydrolyzed to two or more monosaccharide units. Suitable polysaccharides and/or monosaccharides can include (but are not limited to) sucrose, fructose, maltose, cellobiose, lactose, trehalose, lactulose, glucose, ribose, galactose, talose, arabinose, fucose, mannose, xylose, erythrose, starch, glycogen, cellulose, and combinations thereof. The amount of polysaccharide and/or monosaccharide in the microparticle can range from about 0.01% to about 15% by weight (e.g., 0.1-15%, 2-12%, or 3-10%). Thus, the microparticle can include at least about (or no more than about) 0.01, 0.1, 0.5, 1, 2, 3, 4, 5, 6, 7, 8, 9, 10, 11, 12, 13, 14, or 15 weight % polysaccharide and/or monosaccharide, based on the total weight of the microparticle. It should also be appreciated that in some embodiments, the disclosed microparticles lack polysaccharide and/or monosaccharide.

In some embodiments, the disclosed microparticles can comprise any known coating made or used in the art, including (but not limited to) hydroxypropyl methylcellulose, sodium carboxymethylcellulose, cellulose acetate, hydroxypropylcellulose, povidone, cellulose acetate phthalate, methyl hydroxyethylcellulose, ethylcellulose, gelatin, pharmaceutical glaze, plasticizer, hydroxypropyl cellulose, hydroxypropyl methyl cellulose phthalate, cellulose acetate phthalate, polyvinyl acetate phthalate, methacrylic acid copolymer, methylcellulose, polyethylene glycol, polyvinyl acetate phthalate, shellac, sucrose, titanium dioxide, polyvinyl polymers, acrylate polymers, ethyl cellulose, cellulose acetate, wax, zein, alginate, chitosan, or combinations thereof. Thus, the coatings can be hydrophobic, hydrophilic, enteric release (delivery of the API in the intestine with little/no delivery in the stomach), and/or naturally derived.

The disclosed coatings can be of any desired thickness. For example, in some embodiments, the coating comprises about 1-50 weight percent of the total weight of the microparticle, such as about 2-40%, 3-30%, 4-20% or 5-10% of the total weight of the microparticle. In some embodiments, the coating can have a thickness of about 1 μm to about 100 μm, such as about 2-90, 3-80, 4-70, 5-60, 6-50, 7-40, 8-30, or 9-20 μm. However, it should be appreciated that microparticles with thinner or thicker coatings are also included within the scope of the presently disclosed subject matter. In some embodiments, a single coating layer can be used. However, the presently disclosed subject matter also includes embodiments wherein the API core is coated with several layers that are identical or different.

The coatings can be applied to the external surface of the API core using any method known or used in the art. For example, in some embodiments, one or more coatings can be applied using fluidized bed (air suspension) coating, spray application coating, spherification, reverse spherification, electrostatic coating, magnetically assisted impaction coating, vacuum film coating, compression coating, and/or dip coating techniques, as would be known to those of ordinary skill in the art.

As set forth above, in some embodiments, the API material can be dispersed through one or more excipients. The term “excipient” as used herein refers to a compound or composition that is not intended to have medicinal activity. Examples of excipients include (but are not limited to) fillers, pH adjusting agents, preservatives, anti-adhesives (such as talc), plasticizers (such as polyethylene glycol, castor oil, diacetylated monoglycerides, dibutyl sebacate, diethyl phthalate, glycerin, propylene glycol, triacetin, polysorbates, sorbitan esters, and/or triethyl citrate), opacifiers (such as titanium dioxide, talc, aluminium silicate, magnesium carbonate, calcium sulfate, and/or aluminium hydroxide), coloring agents, pigments, surfactants (such as alkali metal or alkaline earth metal salts of fatty acids, polyoxyethylenated oils, polyoxyethylenelpolyoxypropylene copolymers, polyoxyethylenated sorbitan esters, polyoxyethylenated castor oil derivatives, stearates, polysorbates, stearylfumarates, glycerol behenate, benzalkonium chloride, and/or acetyltrimethylammonium bromide) diluents, anti-foaming agents, lubricants, binders, granulating aids, taste modifying agents, and/or glidants that are conventional in the pharmaceutical art. In some embodiments, the excipients are hydrophobic (e.g., waxes or lipids), hydrophilic, enteric-release, and/or naturally derived.

The disclosed microparticles can comprise about 0.1-95 weight percent excipient. Thus, the microparticle can include about 0.1, 0.5, 1, 5, 10, 15, 20, 25, 30, 35, 40, 45, 50, 55, 60, 65, 70, 75, 80, 85, 90, or 95 weight percent excipient.

The API microparticles can be prepared using any method known or used in the art. For example, the microparticles can be prepared using centrifugal extrusion of waxes, lipids, or oils with dissolved or dispersed APIs that are optionally coated in a fluidized bed with a Wurster or powder-coating insert to apply a diffusion barrier and/or enteric coating. In some embodiments, the microparticles can be constructed using spheronization of the APIs by coating inert cores (such as sugar or microcrystalline cellulose spheres) with powder APIs granulated with binders and/or excipients in a high-shear powder-coating fluidized bed and/or Wurster fluidized bed to produce an API matrix particle. In some embodiments, the particles can be further coated in a fluidized bed with a Wurster or powder-coating insert to apply a diffusion barrier and/or enteric coating.

In some embodiments, the disclosed microparticles can have a size of about 1200 μm or less, such as about 10-1000 μm, 25-800 μm, 50-600 μm, 75-500 μm, or 100-450 μm. However, the term “microparticle” is not limited to a particular size and the presently disclosed subject matter can include microparticles with sizes larger and smaller than the ranges recited herein.

In some embodiments, the disclosed API microparticles are spherical in shape. However, the presently disclosed subject matter is not limited and can include microparticles of any desired shape (i.e., spheroid, oblong, cube, pyramidal).

Optionally, the disclosed API microparticles can be stored for an extended period of time in a dried form prior to mixing into a suspending vehicle. For example, in some embodiments, the microparticles are stable in dried form for at least a few months (e.g., 1, 2, 3, 4, 5, 6, 7, 8, 9, 10, or 11 months) or a few years (e.g., 1, 2, 3, 4, or 5 years).

Prior to consumption, the API microparticles are incorporated into a thixotropic suspending media, creating a liquid suspension at rest and at certain temperature ranges (e.g., greater than 50° C. and less than 125° C.). The term “thixotropic” as used herein refers to a shear-thinning property, where a gel or liquid becomes less viscous when shaken, agitated, or otherwise stressed. The term “suspension” or “suspension media” refers to a liquid that includes a dispersion of a component (e.g., an API) that is mixed with, but generally insoluble in, the liquid.

In some embodiments, the thixotropic suspensions can be aqueous-based (e.g., hydrocolloids), lipid-based (e.g., oleogels), or emulsions. The term “oleogel” refers to structured networks of edible oils that exhibit solid-like properties. Although saturated fats and trans fats also display solid-like characteristics at room temperature, these fats are often associated with negative health effects. Oleogels allow for the use of liquid oils that comprise high amounts of healthier unsaturated fatty acids that display solid-like rheological properties when mixed with gelling agents, such as plant waxes (canuba wax, candelilla wax, sunflower wax, rice bran wax, etc.) or food-grade polymers (ethylcellulose, etc.). Thus, oleogels provide desirable characteristics, such as increased viscosity to prevent settling, as well as provide a stabilizing micro-environment for water-sensitive ingredients.

In some embodiments, the suspension media comprises at least one monosaccharide and/or polysaccharide. For example, the suspension media can comprise dextrose, sucrose, fructose, maltose, cellobiose, lactose, trehalose, lactulose, glucose, ribose, galactose, dextrose, talose, arabinose, fucose, mannose, xylose, erythrose, starch, glycogen, cellulose, or combinations thereof at a concentration of about 50 mM to about 500 mM.

Thixotropic semi-solids or liquid suspensions have properties that enable the dissolution or suspension of the API microparticles in a form that is stable until agitated or extruded, at which point the suspension becomes fluid and can be dispensed. In some embodiments, the thixotropic suspensions are formed via molecular self-assembly of cross-linked polymers, causing the microparticles that are agitated with the vehicle to be embedded with a verifiable solution strength and uniform volumetric concentration of ingredients to function as the components in the building of customized formulations. The suspending media behaves as a semi-solid with nearly uniform dispersion of the API microparticles within the 3-D suspension network (e.g., uniform dispersion within about 10%, 5%, 4%, 3%, 2%, 1%, or 0.1% of the mean of random samplings of the dispersion).

The suspending media can include any of the wide variety of thixotropic materials known in the art. In some embodiments, suitable thixotropic suspending vehicles can comprise one or more polyols, lipids, and/or semi-solid media. The term “semi-solid” refers to a composition that is a mixture of liquid and solid phases, having a viscosity of about 40,000-800,000 centipose. In some embodiments, the suspension media can comprise a hydrocolloid or other edible polymer matrix. The term “hydrocolloid” as used herein refers to molecules that are dispersible in water or an aqueous solution. Thus, the suspending media can comprise gelatin, polymeric glycosaminoglycans, agar, carrageenan, alginate, natural gums, carboxymethyl cellulose, xylitol, sorbitol, mannitol, glycerin, pectin, dextran, dextran derivatives, pullulan, xanthan, xyloglucan, starch, hyaluronic acid, guar gum, locust bean gum, gellan, carboxy-methyl-cellulose, acacia gum, propylene glycol, polyethylene glycol, polypropylene glycol, poly(tetramethylene ether) glycol, and/or combinations thereof. In some embodiments, the suspending media can be liquid or semi-solid.

The suspending media can have an acidic pH in some embodiments. For example, the pH of the suspending media can be below about 6, 5, or 4. In some embodiments, the pH of the suspension media can be about 3-4.

The suspension media can have a viscosity in the range of at least about 1-30 centipoise to facilitate suspension of the encapsulated APIs. In addition, settling, diffusion of solutes from the microparticles, and/or agglomeration of the microparticles are also decreased as a result of the viscosity of the suspension media.

The suspension media can include one or more agents or polymer structures (such as ethyl cellulose and/or methyl cellulose) that mechanically and/or chemically preserve the structural integrity of the microparticles, thereby minimizing leakage of the APIs when in suspension. Further, in embodiments wherein the microparticles comprise an outer shell coating, the coating protects the API cores from chemical and physical degradation, allows separation of incompatible APIs or other substances within the suspension, and/or prevents undesirable release of APIs or other substances within the suspension. For example, in some embodiments, less than about 10%, 5%, 4%, 3%, 2%, 1%, or 0.1% by weight of encapsulated API is released into the suspension vehicle, based on the initial total weight of the microparticles.

In some embodiments, the pH of the suspension can be less than 6.0, 5.0, 4.0, or 3.0, depending on the desired release profile of the APIs. Thus, the suspension can have a pH of about 3-6, 4-6, or 5-6. Alternatively, in some embodiments, the pH of the suspension can be greater than 6.0, 7.0, or 8.0, such as about 6-8.

The microparticles can be dispersed in the thixotropic suspension media using methods well known in the art. For example, a customized quantity and list of API microparticles can be blended into the suspension vehicle via a dispersion mill, whisking, homogenization, heat, change in pH, and/or addition of anions or cations to re-solubilize the thixotropic media and then reform it to a final product. During, before, and/or after dispersion of the API microparticles into the suspending media, various components to improve flavor, texture, and/or stability of the suspension can be added. Such components can include (but are not limited to) natural and synthetic flavoring agents (such as corn syrups), food fillers (such as applesauce, fruit purees, and/or hummus), emulsifiers (such as lecithin), preservatives (such as sodium benzoate and/or potassium sorbate), stabilizers (such as proteins, starch, pectin, plant particles, and/or food gums), surface active agents, dispersing agents, sweetening agents, coloring agents, anti-foaming agents, suspending agents, pH regulating agents, buffers, salts, antioxidants, thickening agents (such as xanthan and/or dietary fiber) and/or chelating agents. Such agents can be selected from conventional pharmaceutically acceptable materials as would be known and used in the art.

In some embodiments, the suspension comprises about 30-99 weight % suspension media and about 1-70 weight % microparticles, based on the total weight of the suspension. Thus, the suspension media can be present in an amount of at least about (or no more than about) 30, 35, 40, 45, 50, 55, 60, 65, 70, 75, 80, 85, 90, 91, 92, 93, 94, 95, 96, 97, 98, or 99 weight %, based on the total weight of the suspension. The microparticles can be present in an amount of at least about (or no more than about) 1, 2, 3, 4, 5, 6, 7, 8, 9, 10, 15, 20, 25, 30, 35, 40, 45, 50, 55, 60, 65, or 70 weight %, based on the total weight of the suspension.

In some embodiments, the disclosed suspensions comprise one or more different populations of microparticles that differ from one another in the nature of the API contained within the microparticle, the composition of the coating, and/or the thickness of the coating. In these embodiments, the suspensions can be customized to treat a subject in need of a therapeutically effective amount of a specific combination of APIs. For example, the suspension can comprise a first type of API microparticle wherein the API is a vitamin, and second type of API microparticle wherein the API is a cardiovascular drug, where both APIs are evenly distributed in the suspension media. Further, the concentration of a desired API microparticle in the suspension can vary based on dosage, concentration, and the like. For example, if the appropriate dosage of the API is 10 mg and the microparticles are embedded in the suspension at a concentration of 5 mg/mL, then the equivalent of 2 mL will be administered to the subject.

In some embodiments, the API remains primarily partitioned in the microparticles after elevated temperature pasteurization, food additive pasteurization, or both. The term “primarily partitioned” refers to an amount of at least about 85%, 90%, or 95% remaining in the microparticle instead of migrating into the suspension media. “Elevated temperature pasteurization” and/or “food additive pasteurization” refer to the high temperatures typically exhibited during pasteurization processes. For example, certain dairy and fruit juices are pasteurized at less than 100° C. (e.g., 60° C.−100° C.) for 15-120 seconds (e.g., 15-60 seconds) to eliminate pathogens and extend shelf life.

The term “subject” as used herein refers to a living animal, such as a mammal. Suitable subjects can include (but are not limited to) humans, cats, dogs, non-human primates, horses, pigs, cattle, rabbits, goats, rats, mice, and the like.

The disclosed suspensions have a homogeneity that enables the APIs within the microparticles to be uniformly dispersed but undissolved within the suspension media. For example, in embodiments wherein the APIs have been microencapsulated in protective shells (e.g., coatings), the APIs are kinetically restrained from saturating the suspension vehicle. As a result, the APIs remain unmixed (or nearly unmixed), therapeutically effective, and do not agglomerate after processing and/or prolonged storage, such as for about 15, 30, or 60 days or more.

Thus, the concentration of the API within the liquid suspension can remain in a non-equilibrium state for a prolonged period of time, such as a few days (i.e., 1, 2, 3, 4, 5, or 6 days), a few weeks (i.e., 1, 2, 3, 4, or 5 weeks), a few months (e.g., 1, 2, 3, 4, or 5 months), or longer. In some embodiments, the dissolution of API during storage can be delayed by tailoring the pH and/or viscosity of the suspending media. For instance, an enterically coated microparticle exhibits less dissolution and releases fewer API in acidic suspending media when compared with basic or neutral suspension medias. Further, viscous suspension medias exhibit reduced mixing dynamics and solute diffusion which lessens the release of API from microparticles compared to less viscous suspension media.

Dissolution of APIs can be measured using USP standard protocols, as would be known in the art. Additionally, to compare the dissolution of API microparticles in standard dissolution media versus the thixotropic media disclosed herein, API microparticles are stored for a prolonged period of time in the suspending vehicle. The particles are then separated by filtration, sieving, and/or centrifugation, and the suspending vehicle is collected. The suspending vehicle is then diluted to a non-thixotropic suspension and the API concentration is measured by UV-VIS spectrophotometry or HPLC. The separated microparticles are dissolution tested by standard USP protocols. Alternatively, the dissolution of API microparticles in standard dissolution media versus the disclosed thixotropic media can be measured by collecting API microparticles in an aliquot of the stored delivery vehicle after a prolonged period. The USP Basket Method (Paddle and Basket Method described in U.S. Pharmacopoeia XXII (1990), herein incorporated by reference in its entirety) can then be used to test the dissolution profile of the API microparticles in the suspending vehicle. It should be appreciated that other testing methods known in the art can be used.

As set forth above, the suspension media is thixotropic, which allows the API microparticles to be mixed to an approximately uniform suspension that is also thixotropic, pumpable, and flowable with a reduced settling velocity and reduced flocculation, allowing the suspension to be maintained as nearly uniform for prolonged processing times. As a result, an appropriate compounding process can be used to dispense uniform and accurate doses of APIs and combinations of APIs via volumetric dispensing throughout the period of medication. Advantageously, the liquid suspension comprising the API microparticles withstands high speeds, short-time pasteurization, and packaging processes. For example, in some embodiments, the API microparticle-containing suspensions can be pasteurized without inducing API leakage from the microparticles or a loss of uniformity of the microparticle dispersion.

The disclosed suspensions of APIs can thus be precisely mixed and dosed into palatable oral formulations using, for example, an automated compounding system for personalized treatment of a wide variety of conditions. In some embodiments, the disclosed suspension can be stored in pouches as palatable oral formulations for daily consumption (e.g., eating or drinking) of single or combination therapies for chronic conditions, thereby reducing the pill burden for an extensive patient population. Final volumes of individual single serving sizes of the disclosed suspensions can range from hundreds of milligrams to 100 grams. The disclosed suspension is pumpable and flowable, allowing precise and variable volumetric dispensing of small volumes of the suspension.

Thus, the disclosed API suspensions can be individually formulated for a particular subject, based on the subject's medical history and/or taste preferences. For example, in some embodiments, taste preference-related information can be provided by the subject, such as favorite tastes, textures, and/or dosage size. As such, the APIs of the disclosed suspension are taste masked, allowing for easier dosing of one or more medicines, vitamins, food supplements, etc. Thus, the taste or flavor imparted by the API can be masked or covered to make the suspensions more palatable. In some embodiments, the subject can input the taste preference data into a computing device, or the information can be entered by a medical professional. In this way, a suggested formulation for the suspension media can be determined based on the subject's preferences.

Accordingly, the disclosed system and method can create a customized formulation from data specific to a particular subject that facilitates the single dose oral delivery of one or more APIs, combined in a highly palatable custom mixture with food substances, flavors, and/or textures desired by the subject. In some embodiments, the method includes an automated formulation algorithm that uses correlation and relevance scores to create a list of known and available components for inclusion and proportioned dose of each in the custom mixture, derived from data captured in an individual subject's profile. The taste preference information received can be combined with medical records, test results, and/or genetic tests to compile a composite individual subject profile and preferences score. In some embodiments, the profile can be directly determined by a questionnaire, by online responses to a computer interface, and/or indirectly by other previously captured data sources specific to the subject. The subject's profile can further include information such as the subject's physical attributes and history data including weight, height, sex, age, and health status (e.g., pregnant, active, immobile, and the like). In some embodiments, the algorithm can be based on simple heuristics or more involved statistical methods, such as regression or machine learning.

The individual subject data elements can be provided as parameters of a methods algorithm for ratio metric proportioning of the mass of any component to be included in the disclosed suspensions. The subject profile can further comprise a plurality of other relevant data to further refine the recipe generation, including medical history, family medical history, current nutritional, dietary, and pharmacological product consumption. In some embodiments, the system algorithm of formulation can incorporate results of medical test data (such as blood pressure, blood sugar, and the like), specific medical condition tests, and/or genetic, proteomic, and metabolomics profiles. The subject's profile can then be correlated by a system algorithm with a database of a multitude of consumable components containing correlation scores for their relevance to the data captured in the subject profile. The database of components can include the score for efficacy or applicability to the subject's profile, as determined by available scientific and other publicly available data, such as the peer reviewed data from the National Institute of Health's Office of Dietary Supplements, The Natural Standard (www. Naturalstandard.Com), Beer's List, and other similar available qualified data set. The database on component attributes supplies the method's algorithm with scores for contraindications, strength of scientific evidence relative to effectiveness for specific conditions, and to the subject's profile or to other components, as well as relevant safety precautions of components taken together.

In some embodiments, the method can include cross-correlating with a compound database to determine suitable nutritional compounds, matching compound relevance scores to user factors, checking for drugs taken by the subject dose to individual profile parameters, and/or presenting a recommendation list and amounts to the subject or to a professional. In some embodiments, the subject and/or the subject's physician is allowed to edit and approve of the compound list to edit the selected supplements being added to a dosage, for example. In some embodiments, the method comprises checking for safe levels of compounds after editing to ensure that the compounds are approved and flag any exceptions requiring further review for approval. A customized list dosage with taste preferences can therefore be created for a particular subject.

After the formulation algorithm searches the component database as keyed by the subject's profile for relevance, efficacy, and/or contraindication of all components, the algorithm generates a recommended recipe for the custom API suspension. The recipe includes the recommended dose of one or more APIs as proportioned to the subject's physical parameters and the available dosing information indicated by the manufacturer of the component or by the scientific/public data for that component. The recommended recipe of API components can be presented to the subject and/or medical professional for approval, along with a list of the scores of relevancy, efficacy, and online links to the publication or reference for each component being recommended. Optionally, the subject can edit amounts of the recipe formulation, constrained by safe limitations and contraindications stored in the compounds database, and if desired augment the formulation with other desired compounds or taste, texture, and smell components.

The individual profile can also include flavor, texture, and/or food components that are subjectively chosen to bring the custom mixture to a more palatable and pleasurable state, as may be within the bounds of the volume and media of the custom mixture. The subjective taste data can be used to suggest flavor, texture, color, aroma, and/or other attributes as may be recommended by the algorithm from the available filler components that are compatible with the APIs.

Thus, the disclosed microparticle suspension can include customized formulations of dietary supplements and therapeutics based on the genetic, physical, physiological, and/or medical needs of a particular subject. Accordingly, the disclosed suspensions can be used to treat a medical condition. The disclosed API suspensions can further be beneficial for patients with difficulty swallowing large and/or numerous pills. In addition, subjects with cognitive impairment or those that take a large number of pills would benefit from easy-to-use packaging that facilitates the ability to manage daily intake of APIs. Further, the disclosed system encourages greater consumer compliance with daily intake of key nutritional and pharmacological APIs to obtain desired outcomes. One mechanism by which compliance can be encouraged is by delivering the medical regimen to the patient in a pre-organized and pre-dosed format.

The customized formulations can be packaged using any method known or used in the art. For example, the disclosed API suspensions can be prepared in labelled pouches; plastic, glass, or metal bottles; cans; and the like. In some embodiments, the formulation can be prepared such that dried microparticles are physically separated from the suspension media and the two are mixed prior to consumption by the subject. In some embodiments, the packaging enables the disclosed formulations to be stored or shipped in light or dark conditions, at a variety of temperatures (e.g., 1° C. to 30° C.) for a predetermined period of time (e.g., 1, 15, 30, 60, or more days). Various sizes of the customized formulations can be prepared, such as 0.25-10 ounces, 1-4 ounces, and the like. In some embodiments, the formulation can be packaged as a daily dose of one or more APIs. In some embodiments, it is envisioned that a month's supply of the disclosed formulation can be ordered online, through a healthcare professional, over the phone, by mail, and the like.

Once prepared, a therapeutically effective amount of the disclosed API suspension can be administered to a subject. The term “therapeutically effective amount” refers to the amount of API that will elicit the desired biological or medical response of a tissue, system, or subject. The specific therapeutically effective amount for any particular subject will depend upon a variety of factors including the disorder being treated and the severity of the disorder; activity of the specific API employed; the specific composition employed; the age, body weight, general health, sex and diet of the subject; the time administration, rate of excretion of the specific compound employed; the duration of the treatment; drugs used in combination or coincidental with the specific compound employed; and/or other factors known to those of ordinary skill in the medical arts. In some embodiments, the oral pharmaceutical suspensions can be administered in suitable doses as directed by a physician, veterinarian, or according to the manufacturer's directions. Importantly, the strategy of online ordering with a physician allows the physician to use many closed feedback loops to fine-tune the appropriate dosing. In current systems, the physician must rely on information from the patient that compliance has been high or low. Thus, the disclosed system enables the physician to confirm with much greater certainty that compliance for all drugs in combination has been high. Accordingly, dosing can be fine-tuned in a personalized, closed feedback loop as directed by a skilled physician. Further, the closed loop feedback strategy can serve to open the door for clinical research (i.e., packages with variable dosing of variable combinations are ideally suited to explore a large clinical design space).

In some embodiments, subject and medical professional can work together to decide the specific APIs, nutraceuticals, flavors, and doses that are needed to best treat and satisfy the personalized needs of the subject. A small-batch, automated compounding manufacturer can then produce personalized batches to meet the defined specifications. The subject then receives a shipment of a timed supply (e.g., 1-week supply or a 1-month supply) containing their daily pharmaceutical and/or nutraceutical needs in a semi-solid, palatable, oral formulation in the form of the disclosed API microparticle suspension. The process is repeated for as long as treatment is needed, and thus is ideally suited to the treatment of chronic conditions.

In some embodiments, the disclosed suspension can be configured as a kit. For example, the kit can include microparticles comprising desired APIs in substantially dry form. The microparticles can be provided in a dose that is necessary to treat a particular medical condition. In some embodiments, the microparticles are provided in a dose necessary for modified release (e.g., in gastrointestinal tract fluids). The kit can further include a suspension media and optionally one or more surfactants, colorants, taste-modifying agents, etc. in a dosage sufficient to saturate with the microparticles once the suspension agent the API microparticles have been brought into contact. In some embodiments, the suspension can be configured in pouches. The pouches can be stored and/or delivered at room temperature and/or at cooled temperatures (e.g., about −5 to 8° C.). In some embodiments, the dried microparticles can be prewashed to extract loosely bound API from the particles prior to forming the suspension.

The presently disclosed further enables the in vitro and in vivo release of APIs for absorption after oral administration of the delivery formulation (i.e., the pouched semi-solid dispersion of API microparticles). In some embodiments, the microparticle excipient and/or coating can regulate release of the API through pH-dependent or pH-independent release (e.g., when exposed to gastrointestinal fluid). For example, a pH-dependent coating can function to release an API in the desired areas of the gastrointestinal (GI) tract (e.g., the stomach or small intestine) such that an absorption profile provides at least about 12-24 hours of therapeutic benefit to the subject. When a pH-independent coating is desired, the microparticle excipient and/or coating can be designed to achieve optimal release regardless of pH changes in the environmental fluid (e.g., the GI tract). The presently disclosed subject matter also includes embodiments wherein the microparticle coating and/or excipient releases a portion of the API in one desired area of the GI tract (e.g., the stomach) and releases the remainder of the dose in another area of the GI tract (e.g., the small intestine). Thus, in some embodiments, the microparticles are designed to remain in the small intestine of a subject for a period of at least 5, 6, 7, or 8 hours up to about 24 hours to permit absorption of the API during at least part of the residence time.

Thus, those skilled in the art would be able to design desired in vitro or in vivo API release profiles, such as immediate release, delayed release, extended release, modified release, and controlled release. The term “immediate release” as used herein refers to a dosage form that releases active agent substantially immediately upon contact with gastric juices and will result in substantially complete dissolution within about 1 hour. The term “delayed release” as used herein refers to a release profile in which there is a predetermined delay in the release of the active agent following administration. In some embodiments, the delayed release profile refers to the delay of active agent release until the dosage form reaches the small intestine or colon. The term “extended release” as used herein refers to a dosage form in which active ingredient is released from the formulation at an extended rate such that therapeutically beneficial levels of the active agent are maintained over a prolonged period of time, e.g., an 8 to 24-hour dosage form. The term “modified release” as used herein refers to a dosage form that is slowly and continuously dissolves and/or absorbed in the stomach and/or GI tract over a period of time of about 2 hours or more and/or as a result of environmental factors such as pH, temperature, agitation, concentration of other chemicals (e.g., alcohol), and combinations thereof. The term “controlled release” as used herein refers to a dosage that releases one or more active agents over a prolonged period of time, such as greater than 1 hour.

For example, in some embodiments, the API, excipient material, microparticle coating material, and/or microparticle coating thickness can be selected to achieve a desired release profile of the API. In some embodiments, sustained release systems ensure coverage of the therapeutic need, since the useful plasma concentration of API can be maintained for longer than in the case of immediate-release forms. Furthermore, sustained release systems make it possible to prevent or limit the size and number of API-excessive concentration peaks in the plasma, thereby decreasing the toxicity of the medicinal product and its side effects. Moreover, sustained release systems make it possible by virtue of their increased duration of action to limit the number of daily intakes, thereby decreasing the limitation for the subject and improving the observance of the treatment.

In some embodiments, the disclosed suspension remains in the small intestine of a subject throughout the period required for absorption of the dose of the API. In some embodiments, the disclosed suspension has sufficient mechanical strength to allow the gradual absorption of the API according to a determined and reproducible profile until the dose is fully depleted.

Accordingly, the disclosed system moderates API release after oral administration, in addition to moderating release during the processing and/or storage steps prior to oral administration.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Microparticle Preparation by Centrifugal Extrusion and Fluidized Bed Coating

A microparticle for a cardiovascular combination therapy drug was prepared by centrifugal extrusion and fluidized bed coating. Particularly, vegetable wax was heated to 45° C. to a flowable state. In separate batches, the APIs of Table 1 were individually mixed into the melted wax to produce four 32-day API-in-wax batches. Centrifugal extrusion was used to atomize the API-in-wax dispersion. The flow rate of the dispersion was 50 mL/minute, which streamed onto a disc with a 0.33 radius that was spinning at 1 revolution per second.

TABLE 1
Cardiovascular Formulation of API-in-Wax Core Formulation

| | Daily | 32-Day | API wt/wt | Core |
|---|---|---|---|---|
| | Dose | Batch | in wax | Diameter |
| API | (mg) | (mg) | (payload) | (um) |
| Aspirin | 81 | 2592 | 60% | 300 |
| Atorvastatin | 20 | 640 | 60% | 300 |
| Metoprolol | 12.5 | 400 | 60% | 300 |
| Clopidogrel | 75 | 2400 | 60% | 300 |

The API-in-wax cores were stored as dry particles for one week at temperatures of about 4° C. to 30° C. A film coating of about 3 microns to greater than 5 microns of Eudragit® L100 (Evonik Health Care, Essen, Germany) was applied to the core particles using a powder coating, high shear granulation method with a GRX 35 insert on a VFC Lab 3 Freund-Vector fluid bed dryer (available from Freund-Vector, Marion, Iowa), as set forth in U.S. Patent Application Publication No. 2011/0129530 (incorporated by reference in its entirety herein). Eudragit® L100 was first dissolved as set forth in Table 2 below.

TABLE 2
Dissolving of Eudragit ® L100

| Ingredient | Ratio |
|---|---|
| Eudragit ® L100 | 94 |
| Isopropyl alcohol | 771 |
| Acetone | 515 |
| Deionized water | 64 |
| Triethyl citrate | 10 |

Talc was fed as the binding powder and 94 g of Eudragit® L100 was applied for every 122 g of talc applied (44 wt % Eudragit® L100, 56 wt % talc). Table 3 below describes the resulting API microparticles. The batch size was 1.5 kg of core particles.

TABLE 3
Cardiovascular Formulation Film-Coated
API Matrix Microparticle Cores

| | Film coating of | wt API/wt film- | Thickness shell |
|---|---|---|---|
| API | core wt/wt | coated particle | (μm) |
| Aspirin | 50% | 30% | 20 |
| Atorvastatin | 50% | 30% | 20 |
| Ramipril | 50% | 30% | 20 |
| Metoprolol | 50% | 30% | 20 |

### Example 2

### Preparation of API Microparticle Intermediate Suspensions by Batch Mixing

API microparticle intermediate suspensions for cardiovascular combination therapies were prepared. In a 6 L EKATO mixer (available from EKATO Corporation, Oakland, N.J.), 2 L of a 1% by weight xanthan solution was mixed with a predetermined weight of one type of particles from Table 3 under vacuum at 0.2 atm. 4 batches of intermediate suspensions of API microparticles were produced, as set forth in Table 4.

TABLE 4
API Microparticle Suspension Preparation

| | Batch Volume | Mass particles | g API/mL |
|---|---|---|---|
| API | (L) | (kg) | suspension* |
| Aspirin | 2 | 1 | 0.150 |
| Atorvastatin | 2 | 0.25 | 0.0375 |
| Metoprolol | 2 | 0.25 | 0.0375 |
| Clopidogrel | 2 | 1 | 0.150 |

*Assuming density of 1 g/mL

### Example 3

### Dispensing Personalized Doses of API Suspensions

Personalized doses of multiple microencapsulated API suspensions for cardiovascular (CVD) combination therapies were dispensed using an automated compounding machine (U.S. Pat. No. 9,704,096, incorporated by reference herein in its entirety). Using the compounding machine, a precise and personalized dose of a combination CVD therapy was dispensed and mixed with applesauce (a palatable food filler). Each uniform API microparticle suspension from Table 4 was loaded as a cartridge onto the automated compounding machine. The suspensions were volumetrically dispensed to ensure proper dosing. The dispensing volumes are given in Table 5, illustrating the doses specified for a particular patient, ordered by a doctor via an internet portal. The applesauce flavor was also entered by the doctor, and was selected as a flavor preference by the patient. Citric acid was added to the applesauce to impart a pH of 3.0 in the final food filler-API combination.

TABLE 5
Volume Dispensed for Personalized Dose

| | Daily | 32-Day | Grams | |
|---|---|---|---|---|
| | Dose | Batch | API/mL | mL |
| Cartridge | (mg) | (mg) | suspension | Dispensed |
| Suspension of | 81 | 2592 | 0.150 | 17.28 |
| aspirin in 1% | | | | |
| xanthan media | | | | |
| Suspension of | 20 | 640 | 0.0375 | 17.07 |
| Atorvastatin in 1% | | | | |
| xanthan media | | | | |
| Suspension of | 12.5 | 400 | 0.0375 | 10.67 |
| Metoprolol in 1% | | | | |
| xanthan media | | | | |
| Suspension of | 75 | 2400 | 0.150 | 16.00 |
| Clopidogrel in 1% | | | | |
| xanthan media | | | | |

The final volume of the food filler-API microparticle suspension was 96 ounces, and thus 3.18 L of food filler was added to the API suspensions.

### Example 4

### Pasteurizing and Packaging Personalized Doses of API Suspensions

Multiple personalized microencapsulated API suspensions for cardiovascular combination therapies were pasteurized and packaged using microwave pasteurization and a packaging machine. The food filler—API microparticle slurry was passed through a microwave pasteurizer that raised the temperature of the slurry to 95° C. for one minute. The slurry was then fed to packaging machines that precisely dispensed the slurry into pouches of about 1.5-4 ounces. The pouches were palatable, single-use, one-a-day oral formulations of a personalized combination therapy for cardiovascular disease. The pasteurization conditions are set forth below in Tables 6 and 7.

TABLE 6
Pasteurization Minimum Hold Time Calculations

| | Process Temperature (° C.) | | | | | | | |
|---|---|---|---|---|---|---|---|---|
| pH | 92 | 91 | 90 | 89 | 88 | 87 | 86 | 85 |
| 4.0 | 42.0 | 55.0 | 75.0 | 92.0 | 120.0 | 155.0 | 200.0 | 260.0 |
| 3.9 | 9.0 | 11.0 | 15.0 | 19.0 | 25.0 | 31.0 | 40.0 | 52.0 |
| 3.8 | 4.5 | 6.0 | 7.5 | 9.0 | 12.0 | 15.0 | 20.0 | 26.0 |
| 3.7 | 2.0 | 3.0 | 3.0 | 4.0 | 6.0 | 8.0 | 10.0 | 13.0 |
| 3.6 | 2.0 | 3.0 | 3.0 | 4.0 | 6.0 | 8.0 | 10.0 | 13.0 |

TABLE 7
Pasteurization Minimum Inversion Time Calculations

| | Process Temperature (° C.) | | | | | | | |
|---|---|---|---|---|---|---|---|---|
| pH | 92 | 91 | 90 | 89 | 88 | 87 | 86 | 85 |
| 4.0 | 16.8 | 22.0 | 30.0 | 36.8 | 48.0 | 62.0 | 80.0 | 104.0 |
| 3.9 | 3.6 | 4.4 | 6.0 | 7.6 | 10.0 | 12.4 | 16.0 | 20.8 |
| 3.8 | 1.8 | 2.4 | 3.0 | 3.6 | 4.8 | 6.0 | 8.0 | 10.4 |
| 3.7 | 0.8 | 1.2 | 1.2 | 1.6 | 2.4 | 3.2 | 4.0 | 5.2 |
| 3.6 | 0.8 | 1.2 | 1.2 | 1.6 | 2.4 | 3.2 | 4.0 | 5.2 |

### Example 5

### Food-Grade Preservative Added to Avoid Pasteurization

Food-grade preservatives (such as sodium benzoate and/or potassium sorbate) were added to personalized microencapsulated API suspensions for cardiovascular combination therapies. The food filler—API microparticle slurry was then fed to packaging machines that precisely dispensed the slurry into thirty-two 3-ounce pouches. The pouches were palatable, single-use, one-a-day oral formulations of a personalized combination therapy for cardiovascular disease.

### Example 6

### Preparation of Aspirin Microparticles

Samples of microparticles configured with an API core optionally coated with Eudragrit L30D were prepared. Microparticle type 1 (MP1) comprised 40% aspirin in Sterotex® core (available from ABITEC Corporation, Columbus, Ohio) constructed through melt spray congealing. Microparticle type 2 (MP2) was prepared by adding a 40% Eudagit® L 30D coating to MP1. Eudragrit® L30D is a pH-responsive polymer (pH 5.5-6). Microparticle type 3 (MP3) was prepared by constructing an Atorvastatin core and coating with 40% Eudagit® L 30D.

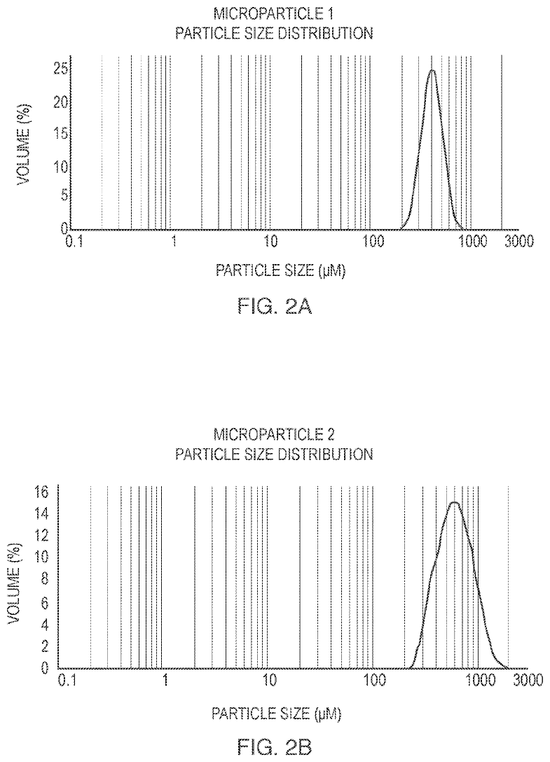

FIGS. 2a-2c illustrate the particle size distribution of produced MPs 1-3, shown as particle size (μm) versus volume (%).

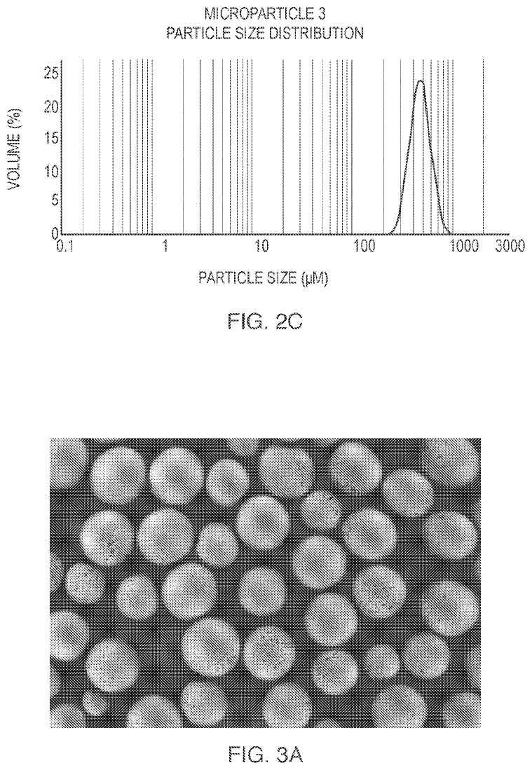

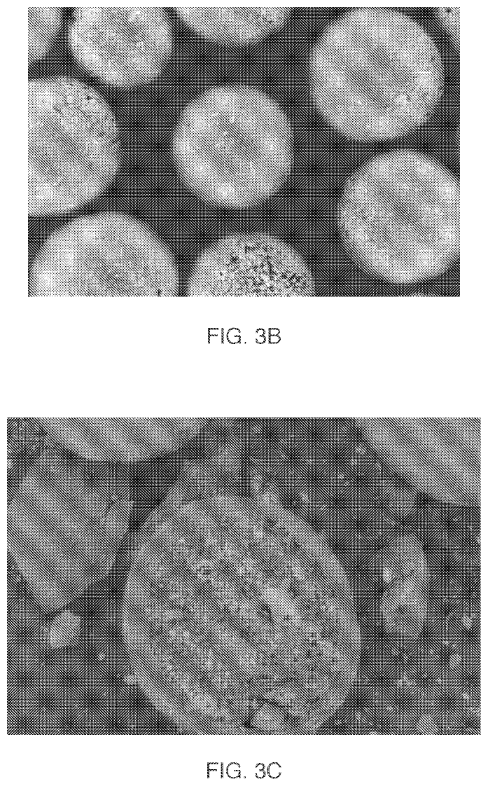

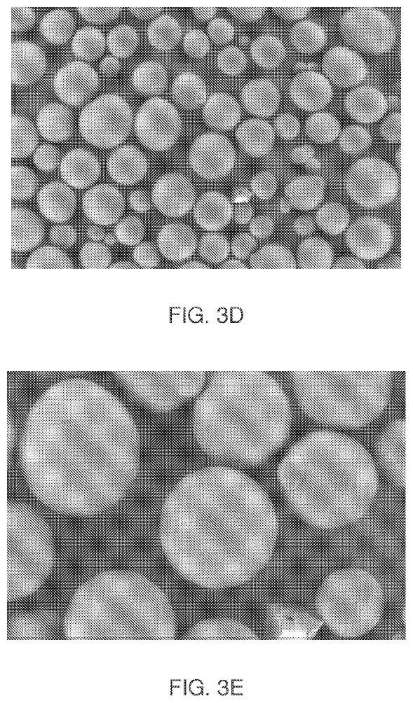

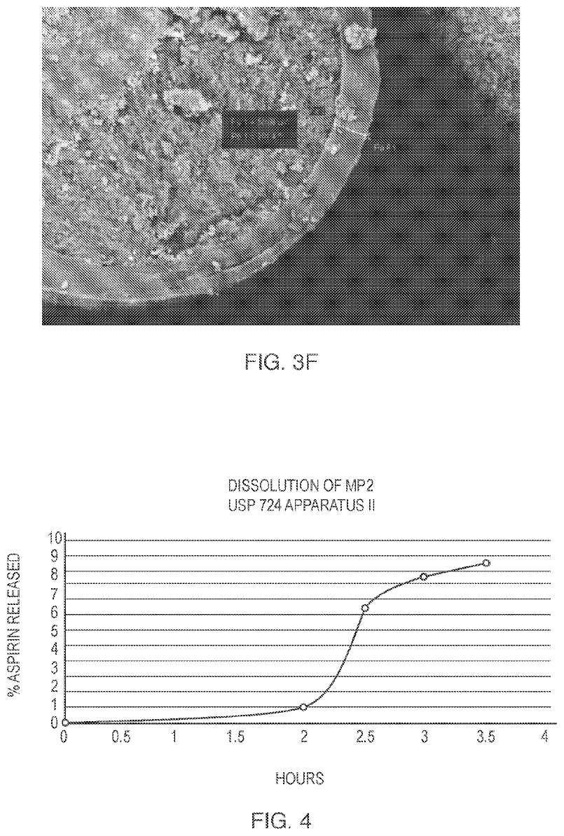

FIGS. 3a and 3b are SEM images of MP1 at 100× and 250× magnification, respectively. FIG. 3c is an SEM image of a 413× magnification of a MP2 microparticle, illustrating the core and outer coating. FIGS. 3d and 3e are SEM images of MP3 at 100× and 250× resolution, respectively. FIG. 3f is a SEM image of a MP3 microparticle at 800× magnification, illustrating the core and coating.

### Example 7

### Dissolution Study of MP2

MP2 microparticles with differing aspirin concentrations were prepared. MP2a and MP2b had an aspirin concentration of 1.5 mg/mL and 20 mg/mL, respectively. The microparticles were cured for 16 hours at room temperature (40° C.) and at 75% RH with no agitation. Citrate media at pH 4 and 3.5 were used as the dissociation medium. 100 μL of the microparticles were taken with replacement of media, as set forth below in Table 8. The percent API released is shown below as the average of duplicate analysis.

TABLE 8
Dissolution of Microparticles 2a and 2b

| | | |
|---|---|---|
| pH Dissolution | Aspirin Conc. | % Released |

| Media | (mg/mL) | 1 hour | 24 hours |
|---|---|---|---|
| 4 | 1.5 | 0.0 | 2.5 |
| | 20 | 0.1 | 2.9 |
| 3.5 | 1.5 | 0.0 | 2.5 |
| | 20 | 0.0 | 2.9 |

MP2 microparticles were further tested for in vitro dissolution based upon USP 724 Method B, using USP Apparatus 2 (paddles) at 100 RMP, 37° C. Sampling included acid stage 2 hours, buffer stage 30, 60, 90 minutes. 1 mL sampled, no replacement buffer used. Filters included dissolution filters 10 um, polyethylene, SunSri PN: 400 104. The samples were tested in triplicate, as shown in Table 9 below. The % aspirin released over time (0-3.5 hours) is shown graphically in FIG. 4.

TABLE 9
MP2 Dissolution Testing, USP 724 Method
pH Values

| Sample No. | Acid Stage (start) | After Buffer |
|---|---|---|
| MP2, | 1.18 | 6.83 |
| replicate 1 | | |
| MP2, | 1.22 | 6.80 |
| replicate 2 | | |
| MP2, | 1.13 | 6.81 |
| replicate 3 | | |

IVR, Acid Stage

| | | | | | Total | | | |
|---|---|---|---|---|---|---|---|---|
| | | | | Amt. | sample | Aspirin | Aspirin | |
| | | Peak 1 | Conc. | Aspirin | wt. | Available | (% | Avg. % |
| Sample | Timept. | Area | (mg/mL) | (mg) | (mg) | (mg) | Release) | Released |
| MP2, | End of | 23197 | 0.001 | 0.72 | 339.61 | 80.49 | 0.9 | 1.0 |
| replicate 1 | Acid | | | | | | | |
| | Stage | | | | | | | |
| MP2, | End of | 24768 | 0.001 | 0.74 | 340.55 | 80.71 | 0.9 | |
| replicate 2 | Acid | | | | | | | |
| | Stage | | | | | | | |
| MP2, | End of | 32878 | 0.001 | 0.87 | 341.33 | 80.90 | 1.1 | |
| replicate 3 | Acid | | | | | | | |
| | Stage | | | | | | | |

IVR, Buffer Stage

| | | | | | Total | | | |
|---|---|---|---|---|---|---|---|---|
| | | | | Amt. | sample | Aspirin | Aspirin | |
| | Timept. | Peak 1 | Conc. | Aspirin | wt. | Available | (% | Avg. % |
| Sample | (min) | Area | (mg/mL) | (mg) | (mg) | (mg) | Release) | Released |
| MP2, | 30 | 203992 | 0.005 | 4.99 | 339.61 | 80.49 | 6.2 | 6.4 |
| replicate 1 | | | | | | | | |
| MP2, | 30 | 209831 | 0.005 | 5.10 | 340.55 | 80.71 | 6.3 | |
| replicate 2 | | | | | | | | |
| MP2, | 30 | 222383 | 0.005 | 5.35 | 341.33 | 80.90 | 6.6 | |
| replicate 3 | | | | | | | | |
| MP2, | 60 | 250037 | 0.006 | 5.90 | 339.61 | 80.49 | 7.3 | 7.7 |
| replicate 1 | | | | | | | | |
| MP2, | 60 | 265906 | 0.006 | 6.21 | 340.55 | 80.71 | 7.7 | |
| replicate 2 | | | | | | | | |
| MP2, | 60 | 280184 | 0.006 | 6.49 | 341.33 | 80.90 | 8.0 | |
| replicate 3 | | | | | | | | |
| MP2, | 90 | 288736 | 0.007 | 6.66 | 339.61 | 80.49 | 8.3 | 8.6 |
| replicate 1 | | | | | | | | |
| MP2, | 90 | 308721 | 0.007 | 7.06 | 340.55 | 80.71 | 8.7 | |
| replicate 2 | | | | | | | | |
| MP2, | 90 | 312586 | 0.007 | 7.14 | 341.33 | 80.90 | 8.8 | |
| replicate 3 | | | | | | | | |

### Example 8

### Microencapsulation of Cardiovascular APIs

As set forth above, MP1 is a microparticle with an aspirin core, MP2 is a microparticle with an aspirin core coated with 40% Eudragrit L30D, and MP3 is Atorvastatin core coated with 40% Eudragrit. MP4 was prepared as a microparticle with a core of Atorvastatin (no coating). The particle size of each API was measured, as shown below in Table 10. There was not enough MP4 produced to run particle size analysis.

TABLE 10
Particle Size Analysis of Microparticles 1-4

| | | API loading | | | |
|---|---|---|---|---|---|
| | | (Theoretical/ | Particle Size (microns) | | |
| Sample | ID | Analytical) | d(0.1) | d(0.5) | d(0.9) |
| 1 | Aspirin core | 40%/— | 300 | 410 | 558 |
| 2 | Aspirin | 28%/23.7% | 368 | 599 | 987 |
| | coated core | | | | |
| | (40% L30D | | | | |
| | coating) | | | | |
| 3 | Atorvastatin | 16%/15.5% | 352 | 479 | 658 |
| | coated core | | | | |
| | (40% L30D | | | | |
| | coating) | | | | |
| 4 | Atorvastatin | 25%/— | — | — | — |
| | core | | | | |

### Example 9

### Short-Term API Release Study of Microparticles 1 and 2

100 mg of each sample was placed in a conical vial and combined with 40 mL of citric acid buffer (2.5 mg/mL), at either pH 6 or 7.3. At desired time points, 1 mL of sample was withdrawn and placed into a microcentrifuge tube and diluted to 5× with citric acid buffer. The vial was replenished with 1 mL fresh citric acid buffer. At the end of the release studies, release was determined by absorbance measurement of the samples at 276 nm compared to a calibration curve (pure aspirin 0.005-0.4 mg/mL) that was validated for accuracy with controls at the low, mid, and high end of the calibration curve (0.015, 0.075, and 0.15 mg/mL). Because the pH of the samples differed, separate calibration curves at a desired pH were used to ensure the detection method was accurate. Tables 11 and 12 illustrate the absorbance at 276 nm for MP1 at pH 6 and 7.3, and the associated controls. Tables 13 and 14 illustrate the absorbance at 276 nm for MP2 at pH 6 and 7.3, and the associated controls.

As shown in the tables below, the standard curve was validated to ensure that the mass of aspirin could be accurately determined during the release studies performed. As shown below, generally this method was valid within 5% error, with the largest error occurring in very low concentration samples (0.015 mg/ml), and only for the pure aspirin cores (MP1).

TABLE 11
Standard Curves for MP1 at pH 6

| | | | | Avg. | |
|---|---|---|---|---|---|
| Sample | Conc. (mg/mL) | Abs. 276 | | Abs.276 | 95% Cl |
| 2 | 0.005 | 0.002 | 0.014 | 0.008 | 0.008499 |
| | 0.01 | 0.015 | 0.035 | .025 | 0.014095 |
| | 0.02 | 0.072 | 0.061 | 0.067 | 0.007436 |
| | 0.04 | 0.127 | 0.133 | 0.130 | 0.007436 |
| | 0.08 | 0.259 | 0.248 | 0.253 | 0.004719 |
| | 0.1 | 0.317 | 0.307 | 0.312 | 0.007574 |
| | 0.2 | 0.620 | 0.629 | 0.625 | 0.006819 |
| | 0.4 | 1.234 | 1.230 | 1.233 | 0.006833 |

| | | | | Avg. | Predicted | | |
|---|---|---|---|---|---|---|---|
| Sample | Conc. (mg/mL) | Abs. 276 | | Abs. 276 | Conc. | % Error | 95% Cl |
| CV | 0.015 | 0.045 | 0.044 | 0.044 | 0.01395 | 6.985 | 0.0004844 |
| | 0.075 | 0.242 | 0.232 | 0.237 | 0.07627 | 1.698 | 0.0070473 |
| | 0.15 | 0.462 | 0.457 | 0.460 | 0.14839 | 1.073 | 0.0035964 |
| R² | 0.9998 | | | | | | |
| m | 3.0900 | | | | | | |
| b | 0.001372 | | | | | | |

TABLE 12
Standard Curves for MP1 at pH 7.3

| | | | | Avg | |
|---|---|---|---|---|---|
| | Conc. | | | Abs. | |
| Sample | (mg/mL) | Abs. 276 | | 276 | 95% Cl |
| 2 | 0.005 | 0.020 | 0.021 | 0.021 | 0.000707 |
| | 0.02 | 0.033 | 0.034 | 0.034 | 0.000276 |
| | 0.04 | 0.108 | 0.108 | 0.108 | 0.001940 |
| | 0.08 | 0.239 | 0.239 | 0.239 | 0.000298 |
| | 0.1 | 0.308 | 0.307 | 0.307 | 0.000575 |
| | 0.2 | 0.639 | 0.641 | 0.640 | 0.000734 |
| | 0.4 | 1.290 | 1.294 | 1.292 | 0.003187 |

| | | | | Avg. | | | |
|---|---|---|---|---|---|---|---|
| | Conc. | | | Abs. | Predicted | % | |
| Sample | (mg/mL) | Abs. 276 | | 276 | Conc. | Error | 95% Cl |
| CV | 0.015 | 0.025 | 0.028 | 0.026 | 0.01347 | 10.1972 | 0.00168041 |
| | 0.075 | 0.229 | 0.229 | 0.229 | 0.07550 | 0.67637 | 6.295E−06 |
| | 0.15 | 0.475 | 0.474 | 0.474 | 0.15040 | 0.27006 | 0.00085233 |
| R² | 0.9994 | | | | | | |
| m | 3.2722 | | | | | | |
| b | −0.01776 | | | | | | |

TABLE 13
Standard Curves for MP2 at pH 6

| | | | | Avg. | |
|---|---|---|---|---|---|
| | Conc. | | | Abs. | |
| Sample | (mg/mL) | Abs. 276 | | 276 | 95% Cl |
| 1 | 0.05 | 0.013 | 0.013 | 0.013 | 0.00036 |
| | 0.01 | 0.027 | 0.027 | 0.027 | 5.613E−05 |
| | 0.02 | 0.054 | 0.0564 | 0.054 | 8.523E−05 |
| | 0.04 | 0.111 | 0.111 | 0.111 | 0.000298 |
| | 0.08 | 0.234 | 0.232 | 0.233 | 0.00185 |
| | 0.1 | 0.280 | 0.280 | 0.280 | 2.772E−05 |
| | 0.2 | 0.562 | 0.562 | 0.562 | 0.000187 |
| | 0.4 | 1.1316 | 1.1345 | 1.113 | 0.002009 |

| | | | | Avg. | | | |
|---|---|---|---|---|---|---|---|
| | Conc. | | | Abs. | Predicted | % | |
| Sample | (mg/mL) | Abs. 276 | | 276 | Conc. | Error | 95% Cl |
| CV | 0.015 | 0.041 | 0.041 | 0.041 | 0.0145 | 3.175 | 1.3859E−06 |
| | 0.075 | 0.211 | 0.211 | 0.211 | 0.0748 | 0.2038 | 6.929E−05 |
| | 0.15 | 0.420 | 0.421 | 0.420 | 0.1492 | 0.5502 | 0.00085926 |
| R² | 0.9997 | | | | | | |
| m | 2.8213 | | | | | | |
| b | −0.01776 | | | | | | |

TABLE 14
Standard Curves for MP2 at pH 7.3

| | | | | Avg. | |
|---|---|---|---|---|---|
| Sample | Conc. (mg/mL) | Abs. 276 | | Abs. 276 | 95% Cl |
| 2 | 0.05 | 0.015 | 0.018 | 0.017 | 0.00215 |
| | 0.01 | 0.031 | 0.034 | 0.032 | 0.00206 |
| | 0.02 | 0.067 | 0.068 | 0.067 | 0.00122 |
| | 0.04 | 0.142 | 0.142 | 0.142 | 0.000478 |
| | 0.08 | 0.285 | 0.283 | 0.248 | 0.001538 |
| | 0.1 | 0.351 | 0.350 | 0.351 | 0.000817 |
| | 0.2 | 0.567 | 0.566 | 0.566 | 0.001122 |
| | 0.4 | 1.381 | 1.381 | 1.381 | 0.000208 |

| | | | | Avg. | Predicted | | |
|---|---|---|---|---|---|---|---|
| Sample | Conc. (mg/mL) | Abs. 276 | | Abs. 276 | Conc. | % Error | 95% Cl |
| CV | 0.015 | 0.051 | 0.051 | 0.051 | 0.0142 | 4.942 | 0.000187 |
| | 0.075 | 0.263 | 0.264 | 0.264 | 0.0758 | 1.114 | 0.00015 |
| | 0.15 | 0.519 | 0.519 | 0.519 | 0.1498 | 0.108 | 7.62E−05 |
| R² | 0.9999 | | | | | | |
| m | 3.4525 | | | | | | |
| b | 0.00176 | | | | | | |

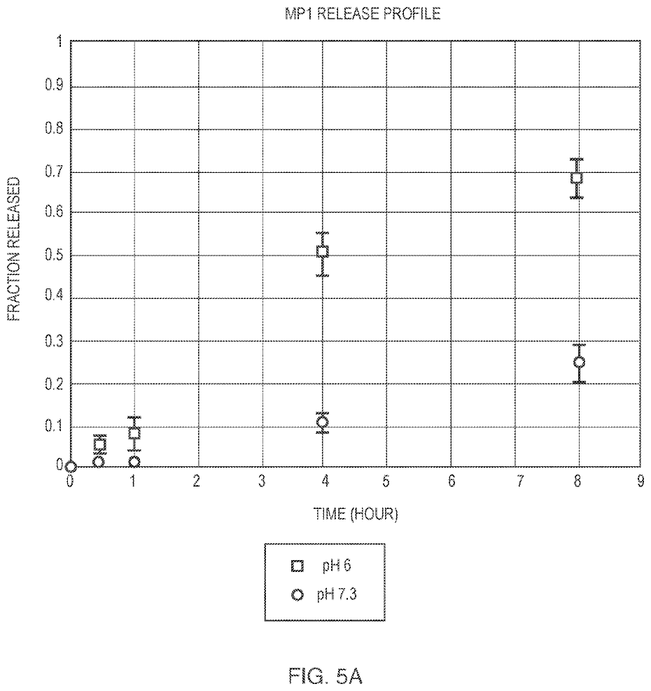

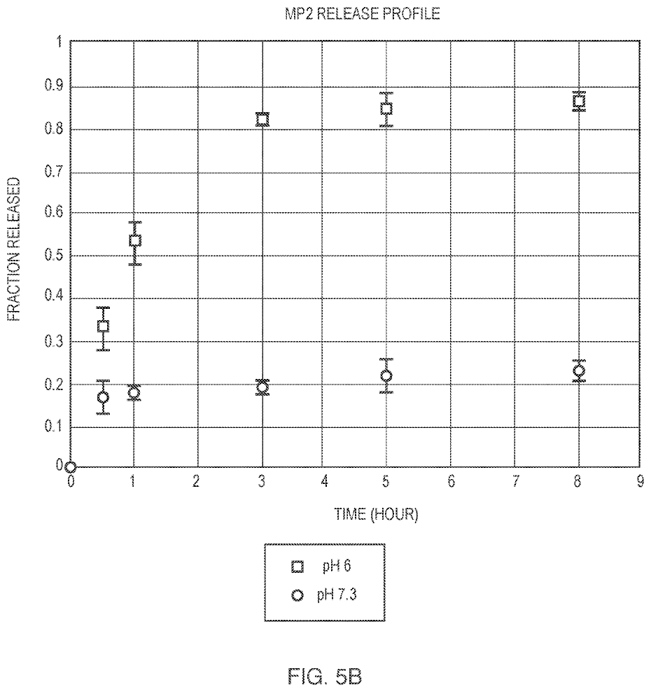

To analyze the results, MP1 samples and MP2 samples were measured by UV-vis at 276 nm, the peak value for aspirin in citric acid buffer (1 mg/mL). Initially, the pure aspirin cores (MP1) were analyzed to ensure release of aspirin would occur in conditions similar to the desired release location (duodenum). The release profiles are given below in Table 15, and are presented graphically in FIGS. 5a and 5b. Release studies were performed over 8 hours to mimic gastric and intestinal emptying consistent with human digestion.

TABLE 15
Release Profiles MP1 and MP2

| | Time | pH 6 Release | pH 7.3 Release |
|---|---|---|---|
| Microparticle | (hr) | (%) | (%) |
| 1 | 0 | 0.00 | 0.00 |
| | 0.5 | 5.13 | 0.58 |
| | 1 | 8.78 | 0.76 |
| | 4 | 50.19 | 10.60 |
| | 8 | 67.80 | 24.57 |
| 2 | 0 | 0.00 | 0.00 |
| | 0.5 | 32.33 | 17.10 |
| | 1 | 51.44 | 18.49 |
| | 3 | 81.16 | 19.67 |
| | 5 | 85.27 | 20.81 |
| | 8 | 86.70 | 22.71 |

As shown in Table 15 and FIGS. 5a and 5b, the aspirin cores (MP1) exhibited a release of about 70% at pH 6 and about 25% at pH 7.3 after 8 hrs. The coated aspirin sample (MP2) exhibited a release of about 90% at pH 6 and about 20% at pH 7.3. The Eudragit® L30D coating is pH sensitive and triggered in the pH 5.5-6 range. There was a discrepancy noted between the samples for release of aspirin observed at pH 6. The discrepancy was apparent at lower time points, e.g. the release of aspirin was 9% and 51% for Microparticles 1 and 2. The discrepancy observed at lower time points can most likely be attributed to a contribution from the Eudragit® coating in absorbance.

Release studies were performed in duplicate. As shown, pH 6 conditions resulted in the release of a higher quantity of aspirin quickly than pH 7.3 conditions.

### Example 10

### Long Term Release API Studies

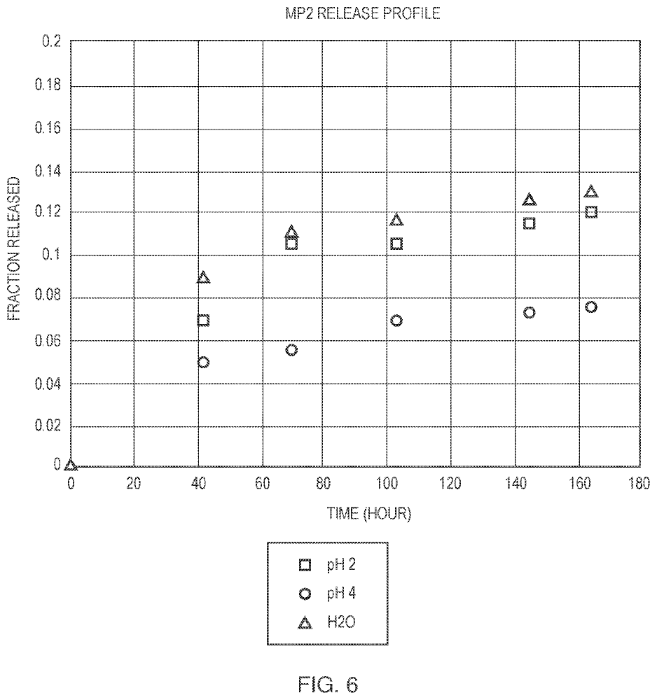

Release studies were performed with MP2 samples to determine optimal pH for long term storage of the particles. Testing was performed at pH 2 and 4 citric acid buffer (10 mg/mL) and in neutral pH (water). The data is shown in FIG. 6 and Table 16.

TABLE 16
Release Profile of MP2

| Time (hr) | pH 2 Release (%) | pH 4 Release | Water Release (%) |
|---|---|---|---|
| 0 | 0.00 | 0.00 | 0.00 |
| 41 | 5.00 | 6.97 | 9.22 |
| 68 | 5.61 | 13.33 | 10.53 |
| 102 | 6.70 | 10.72 | 11.81 |
| 144 | 7.38 | 11.40 | 12.49 |
| 165 | 7.45 | 11.98 | 12.73 |

As shown, there was a substantial difference between prolonged release of aspirin in MP2 at the different pH values. Particularly, there was virtually no difference between samples stored in water and samples stored at pH 4 citric acid, releasing 12.7% vs. 12% after 165 hrs, respectively. However, a substantial difference was noted between pH 2 and pH 4 after 165 hrs. The higher stability is believed to result from the coating providing increased stability at acidic pH compared to neutral pH. Also, an increase in the ionic strength of the storage media at low pH was believed to result in an osmotic gradient, which would provide increased stability at acidic pH.

### Example 11

### NaCl Release API Studies

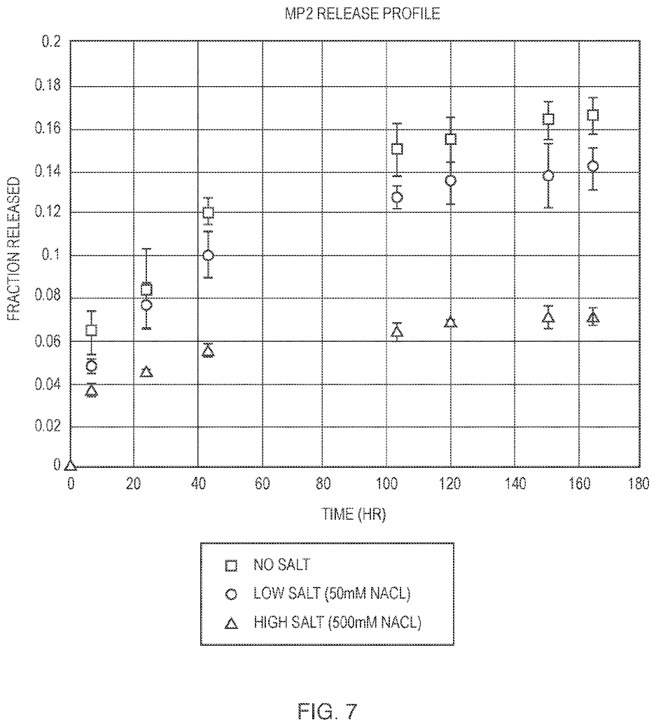

The effect of ionic strength of the storage buffer on prolonged release of MP2 was investigated. To isolate the effect of buffer and ionic strength, release studies were performed in the absence of citric acid. A low salt (50 mM NaCl) and a high salt (500 mM NaCl) condition were tested. The data is given below in Table 17 and is shown graphically in FIG. 7.

TABLE 17
NaCl Release Profile of MP2

| Time | Water % | 50 mM NaCl % | 500 mM NaCl % |
|---|---|---|---|
| (hr) | Release | Release | Release |
| 0 | 0.00 | 0.00 | 0.00 |
| 7.5 | 6.34 | 5.24 | 3.87 |
| 24 | 8.71 | 7.84 | 4.66 |
| 45.5 | 11.91 | 9.96 | 5.48 |
| 102.5 | 14.94 | 12.88 | 6.17 |
| 120 | 15.66 | 13.50 | 6.54 |
| 150 | 16.37 | 13.91 | 6.88 |
| 164 | 16.66 | 14.13 | 6.89 |

As shown, the ionic strength of the buffer in which the particles are suspended substantially affects API release. As the ionic strength of the storage media is increased, the release of aspirin decreases from 16.7% in water to 14.1% in 50 mM NaCl and 6.9% in 500 mM NaCl after 164 hours of release.

### Example 12

### Sucrose Effect Release Studies

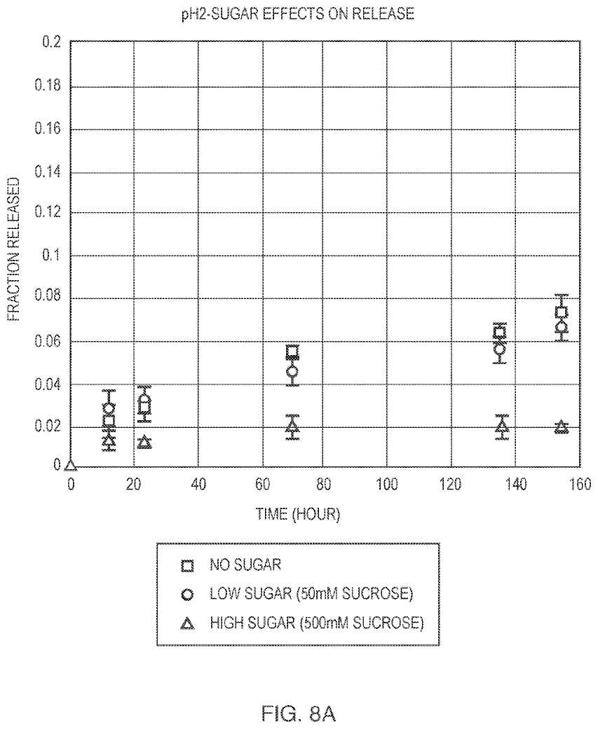

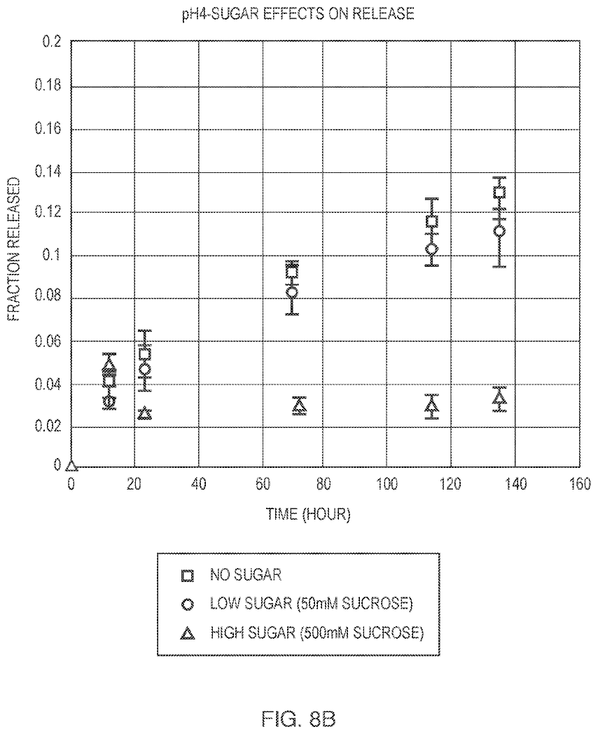

The effect of buffer sucrose concentration on prolonged release of MP2 was investigated. To isolate the effects, the release studies were performed using a 1 mg/mL concentration of citric acid buffer. Low sugar (50 mM sucrose) and high sugar (500 mM sucrose) buffer conditions were tested. The data is given below in Table 18 and is shown graphically in FIGS. 8a and 8b.

TABLE 18
Effect of Buffer Sucrose Concentration on API Release

| | pH 2 | pH 4 | Release (50 | Release (500 |
|---|---|---|---|---|
| Time | Citric Acid | Citric Acid | mM Sucrose) | mM Sucrose) |
| (hr) | (%) | (%) | (%) | (%) |
| 0 | 0.00 | — | 0.00 | 0.00 |
| 12 | 2.06 | — | 2.35 | 1.53 |
| 22.5 | 2.99 | — | 3.24 | 1.46 |
| 72 | 5.36 | — | 4.48 | 1.97 |
| 116 | 6.40 | — | 6.12 | 2.05 |
| 134 | 7.19 | — | 6.78 | 2.03 |
| 0 | — | 0.00 | 0.00 | 0.00 |
| 12 | — | 4.07 | 3.75 | 4.64 |
| 22.5 | — | 5.41 | 4.81 | 2.59 |
| 72 | — | 9.42 | 8.51 | 3.15 |
| 116 | — | 11.54 | 10.44 | 3.09 |
| 134 | — | 12.40 | 10.94 | 3.23 |

Comparing the release profiles within a specific pH, the release studies performed at pH 2 and pH 4 demonstrate a decrease in release over the studied time frame as the concentration of sucrose increases. A 4-fold reduction of aspirin release was observed from the citric acid buffer to the 500 mM sucrose. In pH 2 citric acid, the release decreased from 7.2% (no sucrose) to 2.0% (500 mM sucrose). In pH 4 citric acid, release of ASP decreased from 12.4% (no sucrose) to 3.2% (500 mM sucrose) after 134 hrs. Though a prominent decrease from no sucrose to 500 mM sucrose was observed, there was no statistical difference between no sucrose and 50 mM sucrose. It was therefore concluded that to significantly dampen release of aspirin, it was necessary to have a high concentration of sugar.

Comparing the release profiles across pH 2 and pH 4, it was observed that particles stored at pH 2 released less aspirin compared to particles stored at pH 4. The results were consistent with the pH effect studies and indicate that for maximum retention of aspirin over long-term storage, the use of an acidic storage media was necessary for no/low ionic strength buffers and may be necessary for high ionic strength buffers.

At high sucrose content at both pH 2 and pH 4, release profiles appear to level off. Between the 12 hr and 134 hr time points, it appears that there was <1% aspirin released, implying that a chemical potential equilibrium is established at 12 hrs at the earliest, indicating that the particles were very stable.

### Example 13

### Concentration Effect Release Studies

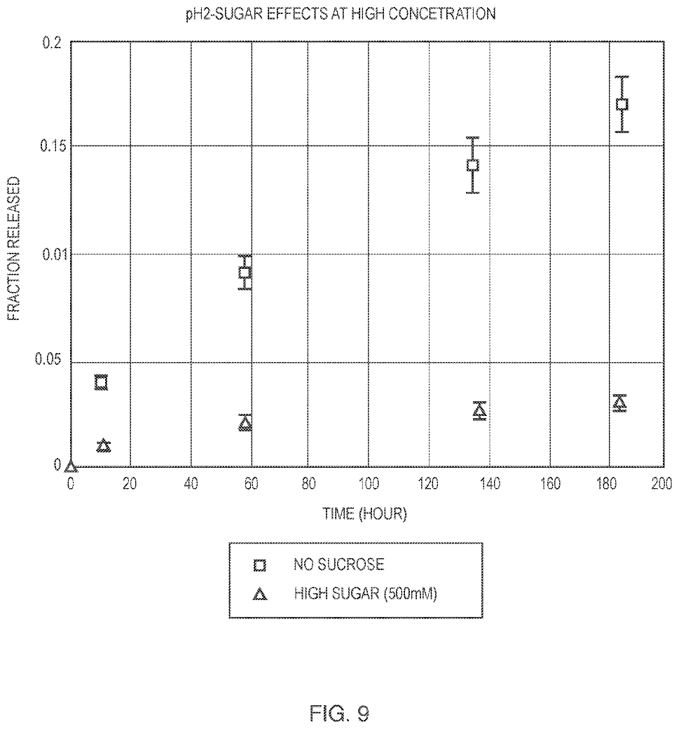

The effect of increasing concentration in the intermediate storage solution for MP2 was investigated at pH 2 and high (500 mM) or low (50 mM) sucrose for 0-187 hours. Based on the release data and known loading data for MP2, a concentration of about 85 mg/mL of particles was necessary to dispense 5 mL of intermediate for accurate dosing of 81 mg of aspirin in vivo. The data is shown below in Table 19 and FIG. 9.

TABLE 19
Concentration Effect on Release of API

| | | 50 mM Sucrose | 500 mM Sucrose |
|---|---|---|---|
| Time (hr) | pH | Release (%) | Release (%) |
| 0 | 2 | 0.00 | 0.00 |
| 12 | 2 | 0.38 | 0.09 |
| 59 | 2 | 0.92 | 0.18 |
| 134 | 2 | 1.49 | 0.27 |
| 187 | 2 | 1.75 | 0.30 |

FIG. 9 illustrates that release profiles at a high concentration (85 mg/mL) of API follows the same physical behavior as the less concentrated system. Particularly, the release of aspirin was dampened drastically by the addition of 500 mM sucrose. In the case of no sucrose, at low concentration, 7.2% of aspirin loaded was released after 134 hours compared to 1.5% of aspirin loaded at high concentration. As observed for high sucrose concentrations, 2.0% and 0.3% of aspirin was released after 134 hours, for low and high concentrations, respectively. One theory is that more aspirin was released than what was soluble. Cloudiness was observed during release, indicating that the aspirin was insoluble in solution. A further cause can be that the cloudiness can be attributed to talc used in sample preparation. The talc can further augment the ionic strength effects seen in previous Examples.

### Conclusion from Release Studies

As set forth in the release studies of Examples 9-13, the most favorable condition for long-term storage of aspirin particles was acidic media, ideally in the range of pH 2-4, and most ideally in the range of pH 2-2.5. It was determined that the addition of sucrose (50-600 mM sucrose or 400-600 mM sucrose) in the acidic media offers additional benefits for long term storage of API particles.

### Example 14

### Long Term Release Study: Aspirin-Xanthan Gum Media

1 mg/ml citric acid buffers were prepared at pH 2 or 4 with 500 mM sucrose by weighing anhydrous citric acid and citric acid into a glass beaker and combining the citric acid with 90-95% of the total volume. The solution was then titrated with HCl to the desired pH before adding water to a final citric acid concentration 1 mg/mL. The solution was then combined with xanthan gum and blended via immersion blending to yield a homogenous thixotropic suspension.

100 mg of MP2 microparticles was weighed and placed into a 50 mL conical vial and combined with 40 mL of the thixotropic suspension (2.5 mg/mL) at either pH 2 or 4 with 500 mM sucrose. At desired time points, 1 mL of sample was withdrawn and placed into a microcentrifuge tube. Because the particles were homogenously suspended, 1 conical vial represented 1 time point, so there was no replenishment of removed buffer. At the end of the release studies, the samples were placed on a Savant SpeedVac Concentrator to remove water from the samples. Samples were then resuspended in the volume of ethanol of the sample that was removed. Ethanol was utilized as the solvent for re-dissolving aspirin because aspirin is soluble in alcohol but xanthan gum is insoluble. Release was determined by measuring sample absorbance at 276 nm and comparing to a calibration curve (pure aspirin 0.005-0.4 mg/mL). The calibration curve was validated for accuracy with controls at the low, mid, and high end of the calibration curve (0.015, 0.075, and 0.15 mg/mL). Because samples can include sugar, separate calibration curves in ethanol with/without sugar were made to ensure method of detection was accurate for the system being studied.

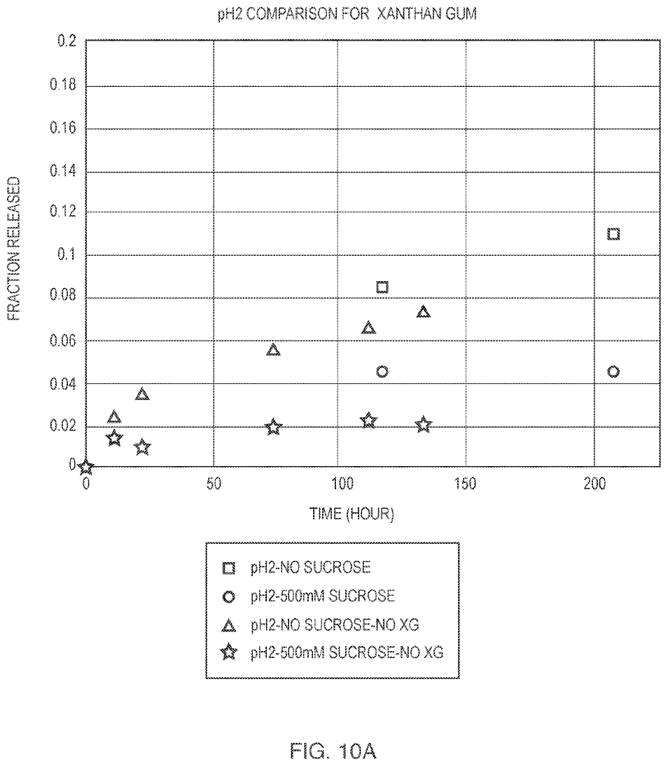

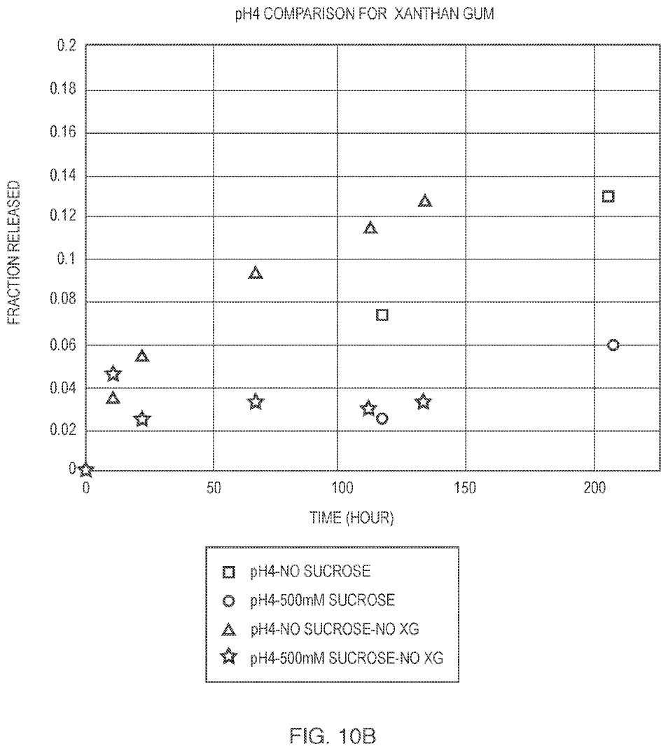

The data is shown graphically in FIGS. 10a and 10b.

### Example 15

### Melt Spray Congealing Trials with Addition of Disintegrant into Core

A disintegrant was added to aspirin and Atorvastatin microparticle cores (50% stearic acid, 10% sodium starch glycolate (SSG) to 40% aspirin; 65% stearic acid and 10% SSG added to 25% atorvastatin). Samples were sieved between 355-500 microns.

FIG. 11a illustrates an SEM image (50×) of a microparticle with a core of 40% aspirin, 50% stearic acid, 10% SSG prior to the addition of water (e.g., dry powder). FIGS. 11b-11e illustrates the microparticle 30 seconds, 2 minutes, 5 minutes, and 10 minutes, respectively, after the addition of water.

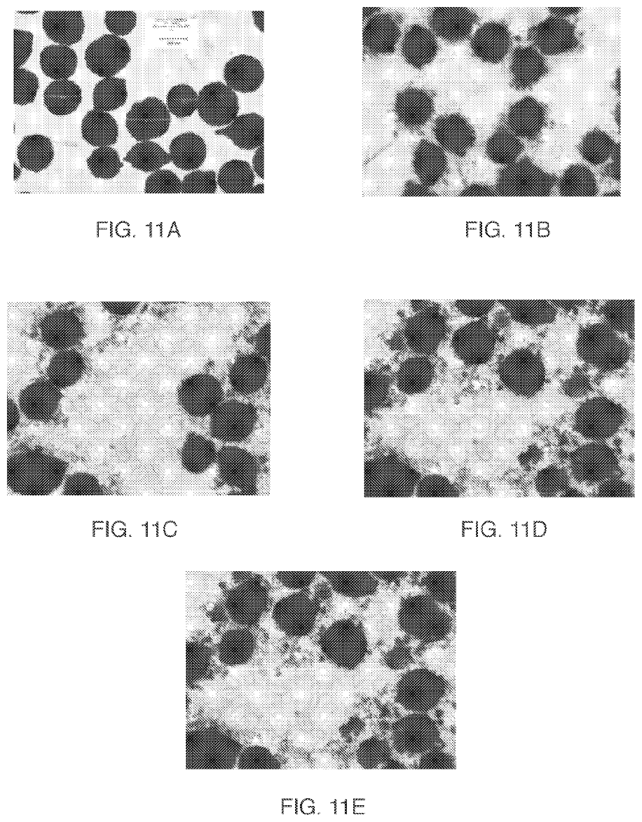

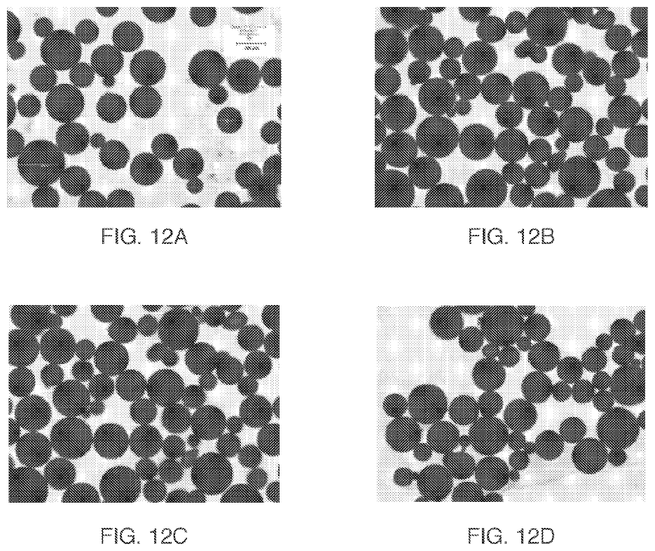

FIG. 12a is a 50×SEM optical photo of a MP1 API (40% aspirin in Steroxtex) prior to the addition of water (dry powder). FIGS. 12b-12d illustrate after 30 sec, 5 minutes, and 10 minutes have passed. As shown in the images there was no appreciable change in shape/surface.

What is claimed is:

1. A suspension for oral consumption, the suspension comprising:
a plurality of microparticles, wherein each microparticle includes a core and an external coating surrounding the core, wherein the core comprises:
about 20-99 weight percent of at least one active pharmaceutical ingredient (API), based on the total weight of the core;
about 0.1-10 weight percent disintegrant, based on the total weight of the core; and
about 0.1-10 weight percent monosaccharide, polysaccharide, or both, based on the total weight of the core;
a thixotropic suspension media, wherein the suspension media is homogeneously distributed with the microparticles;
wherein the core, coating, and suspension media prevent the API from releasing into the suspension until ingestion by a user.

2. The suspension of claim 1, wherein the monosaccharide or polysaccharide is selected from sucrose, fructose, maltose, cellobiose, lactose, trehalose, lactulose, glucose, ribose, galactose, talose, arabinose, fucose, mannose, xylose, erythrose, starch, glycogen, cellulose, and combinations thereof.

3. The suspension of claim 1, wherein the suspension media is a hydrocolloid or oleogel.

4. The suspension of claim 1, wherein the suspension comprises one or more different types of microparticles, each comprising a different API.

5. The suspension of claim 1, wherein the API remains primarily partitioned in the microparticles after elevated temperature pasteurization, food additive pasteurization, or both.

6. The suspension of claim 1, wherein the suspension allows for release of less than about 5% of the API into the suspension while stored.

7. The suspension of claim 1, wherein the API is selected from one or more pharmaceuticals, vitamins, or food supplements.

8. The suspension of claim 1, wherein the microparticle core comprises a coating selected from hydroxypropyl methylcellulose, sodium carboxymethylcellulose, cellulose acetate, hydroxypropylcellulose, povidone, cellulose acetate phthalate, methyl hydroxyethylcellulose, ethylcellulose, gelatin, pharmaceutical glaze, plasticizer, hydroxypropyl cellulose, hydroxypropyl methyl cellulose phthalate, cellulose acetate phthalate, polyvinyl acetate phthalate, methacrylic acid copolymer, methylcellulose, polyethylene glycol, polyvinyl acetate phthalate, shellac, sucrose, titanium dioxide, polyvinyl polymers, acrylate polymers, ethyl cellulose, cellulose acetate, wax, zein, or combinations thereof.

9. The suspension of claim 1, wherein the coating comprises one or more layers.

10. The suspension of claim 1, wherein the suspension media comprises dextrose, sucrose, fructose, maltose, cellobiose, lactose, trehalose, lactulose, glucose, ribose, galactose, dextrose, talose, arabinose, fucose, mannose, xylose, erythrose, starch, glycogen, cellulose, or combinations thereof at a concentration of about 50 mM to about 500 mM.

11. The suspension of claim 1, further comprising at least one additive selected one or more surfactants, colorants, dispersants, preservatives, taste improvers, flavorings, sweeteners, antioxidants, or combinations thereof.

12. The suspension of claim 1, wherein the suspension comprises about 30-99 weight percent suspension media and about 1-70 weight percent microparticles, based on the total weight of the suspension.

13. The suspension of claim 1, wherein the microparticles have an average particle size of between about 100-1000 microns.

14. A method of preparing a suspension comprising a uniform dispersion of microencapsulated active pharmaceutical ingredients (APIs), the method comprising:
receiving health-related information for a subject;
determining an API to treat a medical condition of the subject;
selecting microparticles of a desired API, wherein each microparticle includes a core and a coating, wherein the core comprises:
about 20-99 weight percent of at least one active pharmaceutical ingredient, based on the total weight of the core;
about 0.1-10 weight percent disintegrant, based on the total weight of the core; and
about 0.1-10 weight percent monosaccharide, polysaccharide, or both, based on the total weight of the core;
determining a thixotropic hydrocolloid suspension media;
dispersing a predetermined amount of the microparticles within the suspension media to form a dosage;
wherein the suspension media is solubilized to embed the microparticles and is then reformed as a homogeneously distributed semi-solid suspension; and
wherein the core, coating, and suspension media prevent the API from releasing into the suspension until ingestion by the subject.

15. The method of claim 16, wherein the filler medium is a hydrocolloid or oleogel.

16. The method of claim 16, wherein the suspension allows modified release of at least one API.

17. The method of claim 16, wherein the API is selected from one or more pharmaceuticals, vitamins, or food supplements.

18. The method of claim 16, wherein the coating is selected from hydroxypropyl methylcellulose, sodium carboxymethylcellulose, cellulose acetate, hydroxypropylcellulose, povidone, cellulose acetate phthalate, methyl hydroxyethylcellulose, ethylcellulose, gelatin, pharmaceutical glaze, plasticizer, hydroxypropyl cellulose, hydroxypropyl methyl cellulose phthalate, cellulose acetate phthalate, polyvinyl acetate phthalate, methacrylic acid copolymer, methylcellulose, polyethylene glycol, polyvinyl acetate phthalate, shellac, sucrose, titanium dioxide, polyvinyl polymers, acrylate polymers, ethyl cellulose, cellulose acetate, wax, zein, or combinations thereof.

19. The method of claim 16, wherein the coating comprises one or more layers.

20. The method of claim 16, wherein the microparticles have a particle size of less than about 1000 microns.
